(19) United States
(12) Patent Application Publication
(10) Pub. No.: US 2013/0275316 A1
(43) Pub. Date: Oct. 17, 2013

(54) LIVESTOCK CERTIFICATION APPARATUS AND METHOD

(71) Applicant: Chia-Chi Teng, Provo, UT (US)

(72) Inventor: Chia-Chi Teng, Provo, UT (US)

(21) Appl. No.: 13/829,040

(22) Filed: Mar. 14, 2013

Related U.S. Application Data
(60) Provisional application No. 61/612,894, filed on Mar. 19, 2012.
(60) Provisional application No. 61/659,939, filed on Jun. 14, 2012.

(51) Int. Cl. G06Q 30/00

(52) U.S. Cl. CPC G06Q 30/018
USPC 705/317

(57) ABSTRACT

A system for certification of animals, owners, attendants, venues, enterprises, and events relies on identification, traceability, and evaluation against selected criteria. Data maintained during agricultural production includes production, raising, selling, holding, treating, harvesting products like milk or wool, slaughter, transfer of ownership or venue, and veterinary and pharmacological interventions, whether medicaments, preventives, protocols, treatments, or inspections administered. Record keeping and selection, analysis, and notifications are according to criteria (particularly risks, exposures, or both) specified by any entity requesting or requiring a certification of a condition or lack thereof. Warnings may be triggered and distributed timely. Mobile units avoid traditional deficiencies in data, analysis, risk assessment, and timeliness.

### RELATED APPLICATIONS

This application: claims the benefit of co-pending U.S. Provisional Patent Application Ser. No. 61/612,894, filed on Mar. 19, 2012; and claims the benefit of co-pending U.S. Provisional Patent Application Ser. No. 61/659,939, filed on Jun. 14, 2012; both of which are hereby incorporated by reference.

### BACKGROUND

1. The Field of the Invention

This invention relates to computer systems and software for tracking, analyzing, controlling, and delivering information and, more particularly, to novel systems and methods for certification of animals, personnel and venues associate with livestock operations.

2. The Background Art

Doctors have historically kept patient medical history as a means to diagnose trends and gain a greater historical view over time. Government tracking and industrial databasing of vehicles permit a knowledge of every significant event, and particularly the damaging accidents, to which a car may be subjected. Companies, such as Carfax™ advertise access to such information to potential buyers. A buyer may gain knowledge of such history, and consider it in a purchase decision.

Likewise, books and commentaries from the George Orwell novel, “1984,” and the movie “Many Happy Returns,” to present day political writers and media personalities point out the abilities of the Internal Revenue Service to track virtually all commercial or financial activities of people. Some commentaries point out that modern online financial services bring George Orwell's predictions into possibility.

Similarly, animal identification and traceability have become increasingly important in the agriculture and food industry in recent decades mainly as a way to control quality and to manage animal health information and infectious diseases. Radio-frequency identification (RFID) technology has been used in tracking domestic and wild animals since as early as 1980s.

Industry standards such as ISO 11784/11785 have emerged, encouraging adoption of RFID technology. Commercial systems such as DHI-PLUS, DairyComp, DairyQuest, and the like provide desktop workstation management software. These work in conjunction with RFID-enabled mobile devices and RFID ear tag readers. These operate somewhat like inventory management systems used by retailers, wholesalers, and manufacturers who supply inanimate goods. All the foregoing systems operate as traditional desktop applications. Accordingly, all such systems suffer from the numerous common drawbacks.

For example, they are difficult to upgrade. Upgrading to a new version of desktop application software often leads to compatibility problems and downtime which require extensive and expensive support from the vendors. Support costs can often exceed, and do often exceed, the cost of the software or hardware products themselves. Such systems typically suffer from very long update and release cycles, sometimes lacking support for years. Because of the inconvenience associate with software upgrades, users may be reluctant to upgrade. Even so, new versions usually come out less frequently than once a year or longer. This means new features or bug fixes are usually not available for an unreasonably long period of time, compared to the time at which discovered.

Many fixed locations are difficult to support. Problems encountered on the desktop workstation are usually difficult to diagnose by a support technician over the phone. This is not a great surprise with such limited access. Desktop applications are usually installed in one office location.

For example, farmers who have multiple farm locations or travel on the road have limited access to the data or tools available outside the central office location. Dairy farms are typically 24-hour-per-day and 7-day-per-week (24/7) operations all year around.

It would be an advance in the art to provide to farming and other agricultural managers an ability to manage information, processes, procedures, and all resources generally, from a remote location. Handling feed, water, irrigation, vaccination, testing, treatment, and the like, and the information corresponding thereto, could be a highly desirable benefit. Such functionality could provide economies of multiplexing the time of managers of operations that require “boots on the ground” or “eyes in the field” combined with judgment, management decisions, and travel between physical sites.

Data synchronization is yet another problem. Synchronizing data between mobile field devices and desktop systems available typically requires a physical connection. Again, this is not only inconvenient, but requires a physical presence, and typically happens no more than once a day, often less, and often irregularly. It is not uncommon to find data in the desktop system to be wrong, and usually out of date by a day or more. This was not a problem traditionally.

Even with modern day local area network (LAN) and wide area network (WiFi) technologies, these do not apply to agricultural management of smaller traditional agriculture. Agribusiness systems with layers of management and technology, bring bureaucracy, systems, equipment, and cost. To maintain real-time data update between mobile and desktop applications, requires additional cost for wireless network infrastructure, to say nothing of the practicalities of long absences, battery life on mobile devices, and other issues with cellular networks or the like.

While studies have been published on overall farm management system design and deployment, the failures to meet key operating realities have resulted in little being done to modernize and improve the quality of the herd management software at the heart of any such system.

It would be an advance in the art to provide a more modern service oriented application model using mobile, database, and Web technologies. Great potential for such a system exists if it can develop and deliver new, real-time features.

Likewise, tracking, selecting, evaluating, analyzing, rating and ranking animals, livestock handling, interventions, and venues is not possible on a large scale with great particularity. It would be an advance in the art to make such analysis and its consequences available. Likewise, certification of quality and soundness based on a full history would be of great benefit. A system that could collect, database, analyze, and warn based on such data, including exposure to pathologies could be valuable on a small and large scale.

### BRIEF SUMMARY OF THE INVENTION

In view of the foregoing, in accordance with the invention as embodied and broadly described herein, a method and apparatus are disclosed in one embodiment of the present invention as including a computerized, livestock-management system including a first processor corresponding to an entity exercising central control and responsibility for a first population of livestock. A first memory device, comprising a computer-readable storage medium, is operably connected to the processor and containing a first database comprising first records corresponding to the entity and the livestock. A first input device is operably connected to the first processor, as is a first output device.

A second processor, operating at a remote location and disconnected from the first processor has a second input device operably connected thereto and delivering inputs corresponding to an event at the remote location and a second population of livestock, a subset of the first population, corresponding to the event and located at the remote location. A second output device operably connected to the second processor outputs information corresponding to the second population.

A second memory device, operably connected to the second processor, stores a second database, comprising a copy of the first database, having altered records corresponding to the second population and altered based on inputs from the second input device at the remote location. The second processor is programmed to selectively connect to and disconnect from the first processor based on an availability of an Internet connection therebetween. It may be further programmed to upload altered data, corresponding to the altered records, when connected to the Internet.

Meanwhile, the first processor is programmed to synchronize the first database and second database with respect to one another when the Internet connection is available between the first processor and the second processor. Of course, what can be done in one memory device or processor may be distributed among several, and vice versa.

The system may rely on the first memory device to store a plurality of first modules executable on the first processor, while the second memory device stores a plurality of second modules executable on the second processor.

One may add a third processor (or several more) configured like the second processor to operate with respect to the first processor as a mobile device. A third memory device operably connected to the third processor stores a third database, copied from the first database. That third database is likewise altered in accordance with an event at a third location, remote from the first location (and usually the second) and corresponding to a third population, likewise a subset of the first population. The system may have the first processor programmed to communicate with the second and third processors, when connected respectively to the Internet, which they cannot do in remote locations where livestock events (e.g., administering protocols such as branding, de-horning, treatments, medicines, shots, tests, evaluations, culling, milking, feeding, awaiting gestation, breeding, and so forth).

The system may include first modules such as a synchronizing module executable to broker updates between the first database and the second database. A listening module may be operable to receive over the Internet an upload from the second database by the second processor. The synchronizing module may load into the first processor as a program to broker updates between the second database and the third database.

The second memory device may store, for execution by the second processor, a mobile application programmed to read at least one of the second database, an RFID device, a bar code reader, and a GPS device. The mobile application may be programmed to read the second database, RFID device, bar code reader, GPS device, and the like.

Typically, the mobile application is programmed to read from and write to records in the second database. These may include one or more of an animal record corresponding to an animal in the second population, an event record corresponding to an activity and the second population at the remote location, an attendant record corresponding to an operation conducted at the event, a herd record corresponding to the second population, a herd record corresponding to the first population, a geographical record corresponding to at least one of the animal, the first population, the second population, the event, the attendant, the remote location, or the like.

Records may also include a product record. This may correspond to a product administered at the event. An enterprise record may correspond to ownership of at least one of the first and second populations. A facility record may correspond to a facility corresponding to the enterprise. A personnel record may correspond to at least one of the animal, the first population, the second population, the event, a previous event, the enterprise, the facility, an attendant, or a combination thereof.

The mobile application is typically programmed to read from and write to records in the second database. This may include an animal record corresponding to an animal in the second population, an event record corresponding to an activity and the second population at the remote location, an attendant record corresponding to an operation conducted at the event, a herd record corresponding to the second population, the first population, or both. It may include also a geographical record corresponding to at least one of the animal, the first population, the second population, the event, the attendant, and the remote location.

A product record corresponding to a product may correlate to a material (e.g., drug, medicine, treatment) to be administered at the event. An enterprise record corresponds to ownership of at least one of the first and second populations. A facility record corresponds to a facility corresponding to the enterprise, and may subdivide into various pens, corrals, buildings, functional or otherwise designated enclosures, and so forth. A personnel record may corresponding to any person having a relationship to the enterprise, an attendant, an animal, the first population, the second population, the event, a previous event, the enterprise, or the facility.

In one embodiment, a method for managing livestock, may include providing a first processor and a first memory device comprising a computer readable storage medium storing data and modules executable on the first processor to operate on the data. The data includes a first database, typically including animal records corresponding to a first population of animals and a second population, selected from the first population and located at a remote location away from the rest of the first population.

It will typically include event records corresponding to treatment of the second population administered at the remote location, previous treatments, and so forth. Likewise, geographical records may include maps, GPS coordinates, names, addresses, and unique identifiers corresponding to any previous treatment or event corresponding to an animal of any population.

An enterprise record corresponding to ownership of the first population, and facility record corresponding to at least one of the treatment and the previous treatment, may be correlated with product records corresponding to products administered to animals at any place or treatment event. Records may identify attendants such as veterinary staff and a care staff at treatments. Reports may be saved corresponding to any population, animal, treatment planned or completed, any product, enterprise, or event.

Typically a mobile device processor in a mobile device is used remotely from and disconnected from the home or base processor, typically a base server. Storing a mobile application and a copy of the database the mobile device is connected to load the mobile application. Conducting an event (e.g., treatment, test, action, etc.) is based on presentation by the mobile application of old information from the copy of the database and results in uploading to the copy of the database, by the mobile application, new information corresponding to the subject population and the event.

In another embodiment, a method may include connecting the second processor to the first processor when a connection is available over the Internet. This is followed by brokering, by the first processor, the updating of the first database with the new information from any outstanding copies the database sought to be uploaded and synchronized.

Other mobile devices may have their own (e.g., second or third copy) of the database distinct from the first copy of the database. These may also operably disconnect from the first processor when moved to operate in remote locations. Each may make additions to their copy of the database. Thus, uploading from all these mobile devices their additions to the database will require brokering for priority, hierarchical control, timeliness, and so forth. Each remote or mobile device and its mobile application hosted thereon may interact with the server in the same way, and operate independently at remote sites likewise.

In one method for managing livestock, one may create a database on a first computer readable storage device. A first application on a first processor operably connected to read from and write to the database establishes a connection between the first processor and a second processor over the Internet. It downloads a first copy of the database to the second processor, followed by moving the second processor to a remote location functionally inaccessible to the Internet.

Executing on the second processor is a mobile application accessing the copy. Attendants conducting an event (e.g., administering a protocol such as counting, culling, breeding, treating, inoculating, branding, calving, etc.) with livestock operate at the remote location, and based on animal information drawn from the copy, hosted on the mobile device. Uploading information from the event, and corresponding to the affected population (e.g., herd, group, etc.) results in a modified database. Updating the copy, through an input device operably connected to the second processor, event data corresponding to the livestock and the event may be input by hand, by RFID readers, by GPS readers, and the like, all at the same time, that is, at the event.

The mobile device must determine, by testing by the second processor, when the Internet again becomes available for access by the second processor. It may then connect to the Internet, in response to the test, and begin uploading to the first processor all, or at least a portion, of the modified database reflecting the event linked to the livestock, attendants, medicine, products, and so forth. In general, a protocol may include any test, evaluation, treatment, medicine, re-grouping, separating, quarantine, or other an animal husbandry activity.

Inputting at the event, into the modified database, by the second processor, identification of individual animals of the livestock and identification of actions will reflect the protocol, and all attendant parameters desired, including time, place, facility, animal, attendant, and so forth. Reconciling is done by the first processor accessing and brokering data to be entered into the database to create a modified database. Downloading to the second processor a new database may be done at any time when a connection is available.

In one embodiment, creating a database on a server, comprising software operating on a first processor and a first computer readable storage medium, may be followed by downloading to a mobile device, comprising a second processor operably connected to a second computer readable storage medium, a copy of the database. Then, conducting an event may comprise applying any protocol to a population of livestock, remote from the server, at a location wherein the mobile device lacks access to the Internet and the server.

Conducting the event is based on livestock data in the database as copied into the mobile device, which then modifies the copy by uploading thereto new data reflecting event data linked to the livestock data and corresponding the event to livestock subjected to the protocol. Thus, when moving the mobile device after the event, eventually one will again be able to access the Internet. This is still remote from the server but over the Internet. Thus, one may then update the database with the new data from one or more mobile units whenever access to the Internet becomes available to the mobile devices.

### BRIEF DESCRIPTION OF THE DRAWINGS

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a schematic block diagram of a computer system connected in an internetwork comprising a network local to the computer, containing various localized nodes, and a server, as well as a router connecting to another network as an internetwork;

FIG. 2 is a schematic block diagram of a communication architecture of desktop devices, mobile units, and servers, interacting with a database and software applications in accordance with the invention to track livestock activities and conditions in order to provide operational support and communicable disease transmission certification;

FIG. 3 is a schematic block diagram of web access by a web server, a database server, and a synchronization module for maintaining synchronization of a database, hosted on a central computer, as well as being uploaded and updated to and from various mobile units;

FIG. 4 is a schematic block diagram of one embodiment of a database in accordance with the invention, illustrating various of the operational engines of the database and records stored in the database;

FIG. 5 is a schematic block diagram of a mobile application system, including various modules for execution on a processor of a mobile unit;

FIG. 6 is a schematic block diagram of the livestock manager mobile application illustrated in FIG. 5;

FIG. 7 is a schematic block diagram of a process for executing on a server in accordance with the invention;

FIG. 8 is a schematic block diagram of a process executing on a mobile device in a system in accordance with the invention;

FIG. 9 is an illustration of a facility, containing various holding areas or containment areas, as well as crop production areas, feed stores, animal holding and processing areas, and the like, superimposed with data illustrating historical locations of events corresponding to a particular head of livestock;

FIG. 10 is a schematic block diagram of one embodiment of a data record for animals representing examples of data fields from which the visual presentation of FIG. 9 may be created;

FIG. 11 is an image of a screen shot illustrating one example of a template corresponding to a livestock animal record;

FIG. 12 is a schematic block diagram of various modules operating within a system in accordance with the invention, and stored in memory, to be fed into a processor for execution in order to execute the functionality of a system in accordance with the invention;

FIG. 13 is a schematic block diagram of a system of software modules hosted on one or more processors for effecting compliance with regulations, certifications, permits, marketing, packing, veterinary, or pharmaceutical protocols using the herd management system in accordance with the invention;

FIG. 14 is a schematic block diagram of a process for tracking, evaluating, predicting, reminding, and recording the implementation of protocols and exposures of animals to treatments, events, contacts, and the like;

FIG. 15 is a schematic block diagram of a process for certification and notification as well as remediation of animals and animal products using the herd management system in accordance with the invention; and

FIG. 16 is a schematic block diagram of a process for using certification to document or verify the compliance of an animal or population with a criterion demanded by certification, regulating, permitting, or the like.

### DETAILED DESCRIPTION OF THE PREFERRED EMBODIMENTS

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings, wherein like parts are designated by like numerals throughout.

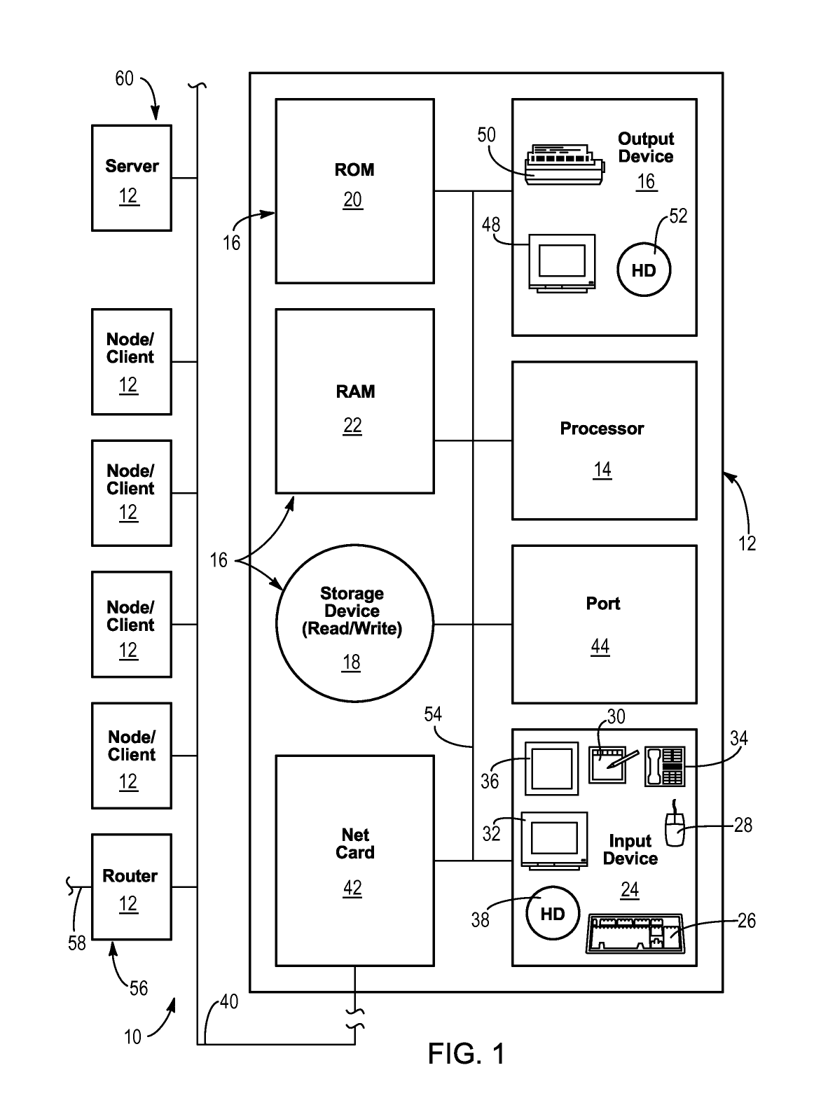

Referring to FIG. 1, an apparatus 10 or system 10 for implementing the present invention may include one or more nodes 12 (e.g., client 12, computer 12). Such nodes 12 may contain a processor 14 or CPU 14. The CPU 14 may be operably connected to a memory device 16. A memory device 16 may include one or more devices such as a hard drive 18 or other non-volatile storage device 18, a read-only memory 20 (ROM 20), and a random access (and usually volatile) memory 22 (RAM 22 or operational memory 22). Such components 14, 16, 18, 20, 22 may exist in a single node 12 or may exist in multiple nodes 12 remote from one another.

In selected embodiments, the apparatus 10 may include an input device 24 for receiving inputs from a user or from another device. Input devices 24 may include one or more physical embodiments. For example, a keyboard 26 may be used for interaction with the user, as may a mouse 28 or stylus pad 30. A touch screen 32, a telephone 34, or simply a telecommunications line 34, may be used for communication with other devices, with a user, or the like. Similarly, a scanner 36 may be used to receive graphical inputs, which may or may not be translated to other formats. A hard drive 38 or other memory device 38 may be used as an input device whether resident within the particular node 12 or some other node 12 connected by a network 40. In selected embodiments, a network card 42 (interface card) or port 44 may be provided within a node 12 to facilitate communication through such a network 40.

In certain embodiments, an output device 46 may be provided within a node 12, or accessible within the apparatus 10. Output devices 46 may include one or more physical hardware units. For example, in general, a port 44 may be used to accept inputs into and send outputs from the node 12. Nevertheless, a monitor 48 may provide outputs to a user for feedback during a process, or for assisting two-way communication between the processor 14 and a user. A printer 50, a hard drive 52, or other device may be used for outputting information as output devices 46.

Internally, a bus 54, or plurality of buses 54, may operably interconnect the processor 14, memory devices 16, input devices 24, output devices 46, network card 42, and port 44. The bus 54 may be thought of as a data carrier. As such, the bus 54 may be embodied in numerous configurations. Wire, fiber optic line, wireless electromagnetic communications by visible light, infrared, and radio frequencies may likewise be implemented as appropriate for the bus 54 and the network 40.

In general, a network 40 to which a node 12 connects may, in turn, be connected through a router 56 to another network 58. In general, nodes 12 may be on the same network 40, adjoining networks (i.e., network 40 and neighboring network 58), or may be separated by multiple routers 56 and multiple networks as individual nodes 12 on an internetwork. The individual nodes 12 may have various communication capabilities. In certain embodiments, a minimum of logical capability may be available in any node 12. For example, each node 12 may contain a processor 14 with more or less of the other components described hereinabove.

A network 40 may include one or more servers 60. Servers 60 may be used to manage, store, communicate, transfer, access, update, and the like, any practical number of files, databases, or the like for other nodes 12 on a network 40. Typically, a server 60 may be accessed by all nodes 12 on a network 40. Nevertheless, other special functions, including communications, applications, directory services, and the like, may be implemented by an individual server 60 or multiple servers 60.

In general, a node 12 may need to communicate over a network 40 with a server 60, a router 56, or other nodes 12. Similarly, a node 12 may need to communicate over another neighboring network 58 in an internetwork connection with some remote node 12. Likewise, individual components may need to communicate data with one another. A communication link may exist, in general, between any pair of devices.

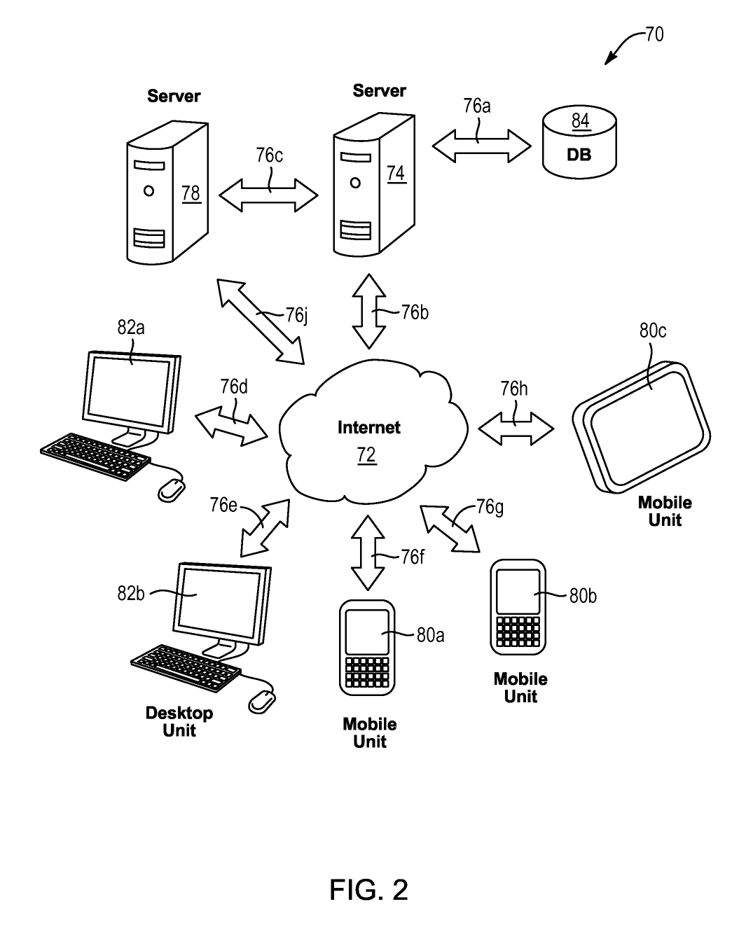

Referring to FIG. 2, an apparatus 70 in accordance with the invention may be hosted on a system 10 as described with respect to FIG. 1. For example, in the illustrated embodiment, the Internet 72 may receive and transmit to and from various modules hosted in various hardware, instructions, data, and the like.

In the illustrated embodiment, a database server 74 communicates through the Internet 72 to various other handheld modules stored in handheld devices 80. In general, a database server 74 connects over the Internet 72 by various links 76, or connections 76 to various mobile devices 80, desktop devices 82, and including other servers 78. For example, the server 78 may operate as a web server, a router, or the like. In general, a computer 78 loaded with server software, may operate as a web server 78, as well as a router 78. Accordingly, the router 56 of FIG. 1 may be embodied in the software hosted on a computer 78.

In general, the mobile units 80 (the trailing letter after each reference numeral reflects a specific instance of the item identified by the reference numeral) for example, a mobile unit 80a, may be a handheld personal digital assistant (PDA 80), a tablet 80, such as the mobile unit 80c, or various other devices.

Meanwhile, a desktop unit 82, may be located in a central office. Typically, a desktop unit 82 may be at a headquarters. In general, the mobile units 80 may be operational in the field. Typically, a handheld device 80 is preferable, because size, space, accessibility, and the like are at a premium. Likewise, there often is no place in the field to set a mobile unit 80.

A mobile unit 80 may be clipped, holstered, or otherwise tethered to a user, set on a dashboard, fender, tailgate, or the like of a vehicle, connected to a fence post or fence, set on a piece of equipment, or the like. Thus, a tablet 80c or a PDA 80a or multiple PDAs 80a, 80b may be used for units in the field in order to document, instruct, record, and otherwise coordinate livestock operations in the field.

Meanwhile, the desktop units 82 may be deployed in an office, in a hotel room, at a regular business location, or the like. For example, police and emergency organizations often deploy desktop units, typically in the form of laptop computers, in vehicles. However, they are permanently anchored, do not move with the user, but remain in the vehicle.

In a system 70 in accordance with the invention, the desktop units 82 may be distributed at different locations. For example, the desktop unit 82a may be located at the office, while a desktop unit 82b may be a computer at a hotel, a laptop of a traveling executive, or the like. Thus, each has the ability to rest permanently on a supporting surface, and is not actually in the field where it may be damaged, where there is no place to support it, or where it may be exposed to the rigors of the operating environment of a livestock operation.

In general, the database 84 is located at a central location, such as a headquarters or operational headquarters of a particular entity. Entities may include owners, companies, corporations, ranching operations, dairy companies, and the like. Typically, an entity may own multiple facilities, and facilities may have multiple operational areas or holding areas in which livestock is held.

For example, barns, corrals, pens, chutes, aisles, milking parlors, branding facilities, coops, sheds, and the like may all exist as holding areas or containment areas within a facility. Thus, the database 84 is typically hosted at a single location, and is accessed by a server 74 responsible therefor.

Similarly, the database server 74 may also communicate to a web server 78 linked to the Internet 72 and other computers 12 that may need to access various information over the web. Accordingly, the server 78 may be a computer 78, or may host software for servers, routers, both, and other software. Nevertheless, in general, a server 74 needs to host various software for managing and interfacing with the database 84.

Meanwhile, each unit 80, 82, connected as part of the system 70, may download a local copy or cache of the database 84. Thus, any personnel operating in the field, when having access to a mobile unit 80c connected through the Internet 72 to the server 74, may upload or download to the database 84. Meanwhile, in the absence of a connection 76b to the server 74, data may be uploaded or downloaded from input and output devices associated with the mobile unit 80, or with a desktop unit 82 to the database copy or cache within the remote unit 80, 82.

Generally, the web server 78 will be responsible for hosting a user interface, communicating information over the web that has been extracted from the database 84, formulating queries and forwarding them to the database server 74 in order to obtain information, and the like. Typically, one may think of the web server 78 as filling more of an administrative function in order to off load the demands on the database server 74.

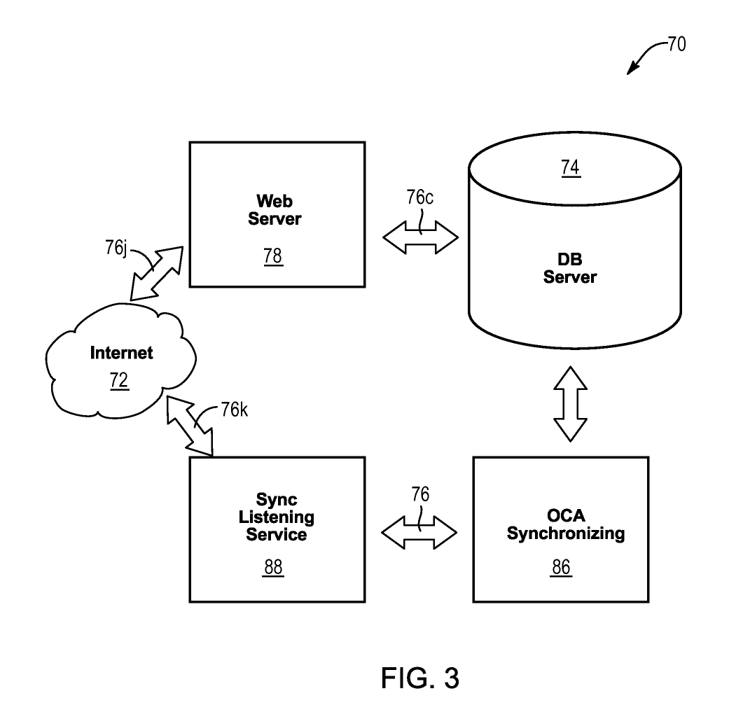

Referring to FIG. 3, while continuing to refer generally to FIGS. 1-12, in one embodiment of a system 70, the web server 78 may maintain a connection 76C to the database server 74 and a connection 76j to the Internet 72. However, hosted in the computer 74, may be other software. For example, an occasionally connected application (OCA) 86 may be responsible for synchronizing between the database 84 managed by the database server 74. The OCA 86 may be configured in any suitable fashion, including those documented in the literature associated with occasionally connected application software from various developers, suppliers, and so forth.

In the illustrated embodiment, a listening service module 88, operates in conjunction with the OCA module 86 by providing a listening service 88 connected to the Internet by a suitable connection 76k. The responsibility of the listening service module 88 is to communicate with the mobile units 80, when the mobile units are accessible. That is, for example, the mobile units 80 are typically, even very often, inaccessible to any Internet connections 76. Accordingly, they must operate independently for extended periods of time, after which, they may upload their contents, download content from the database 84, through the database server 74, or both at periodic connection times.

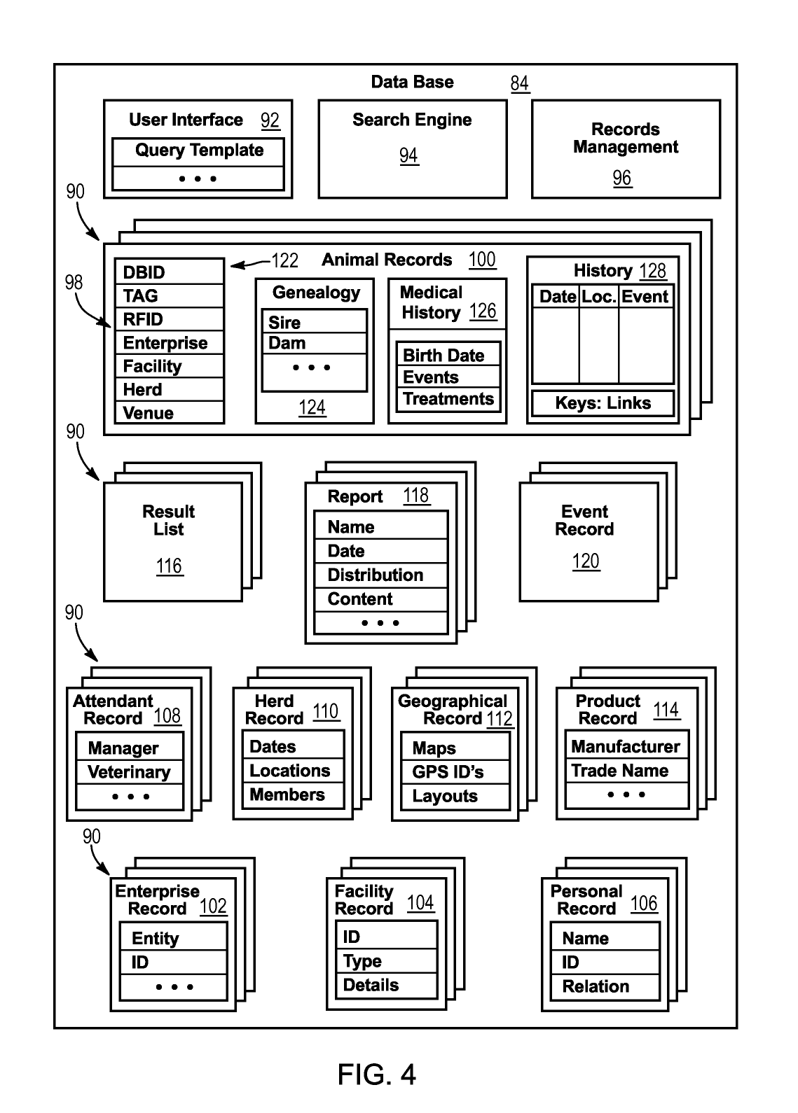

Referring to FIG. 4, a database 84 may include several engines 92, 94, 96, responsible for the administrative functions of the database 84. For example, a user interface engine 92 may be responsible to develop, provide, coordinate, communicate with, and otherwise provide the protocols, templates, graphical user interfaces, and the like to a browser, application, client, or the like connected through the Internet 72 to the database 84 through the database server 74. Typically, a query template will be included, which may be uploaded to a browser, interface with a browser, control a browser, or the like.

Similarly, a search engine 94 may be responsible for processing queries and uploading the resulting search report of information stored in the database 84. Similarly, an administrative module 96 or records management module 96 may execute on a processor 14 in a computer 12 on a connection 76 through the Internet 72 in order to provide records management of various records 90 in the database 84. For example, uploading, downloading, cleaning up, identifying, directing, checking for errors, and so forth may be administrative functions.

The informational heart of a database 84 is the collection of records 90. Just as the search engine 94 operates as the heart of the executable functionality in a database 84, the records 90 actually constitute the database proper, or the records 90 containing information that will be updated, stored, searched, uploaded, reported, and so forth.

In the illustrated embodiment, the records 90 may include, for example, various fields 98. Often, a relational database 84 may be made up of records 90 constituted by tables 90 in which each row represents a specific record 90, and each column represents a field 98 in the record 90. In relational databases, as known in the art, key fields 98 represent fields shared between tables, and thus linking the records 90 of one table to the records 90 of another table.

In general, an animal record 100 will include identifying data 122. In addition, genealogy data 124 is important for various reasons, including breeding, production management, tracking, and so forth Likewise, medical history data 126 and general complete history 128 may be important.

For example, the identification data 122 may include the database identifier, or the number in the database by which the record 90 is known. This may be arbitrarily assigned, and referenced primarily, or entirely, by the database 84 itself. Meanwhile, a tag identifier, such as a visible number on an ear tag or the like, as well as a radio frequency identification tag number (RFID), may be included. Enterprise data, including the name of the enterprise that is the owner of the livestock, a facility identifier, a herd identifier, or the like may be part of a record 90. A herd may be any size of subset from the entire owned population of animals to any small group separated for any particular reason, such as gestation, culling, treatment, or the like. Each may be designated as a herd or population as a temporary identifier, permanent identifier, or the like.

Meanwhile, venue data such as a particular corral, a facility, a pen, a subsystem or subordinate containment or corral, or the like may identify venue. That is, in general, it may be valuable to identify venue by more than a single designation. Multiple levels of venue may be tracked. Likewise, herd identification may be tracked through multiple levels. Herd levels may all be included in the herd identifiers. Thus, the exact location by venue, and by group of livestock may all be considered, at any level of granularity desired to be stored.

By the same token, enterprise information may include enterprise information down to any level management, including a single location and a single individual, or up to the headquarter level of the owner of the enterprise, headquarters location identifier, or the like.

Typically, productivity of livestock, regardless of the purpose for their being managed, pedigree may be tracked. Accordingly, a genealogy module 124 may maintain information regarding sire, dam, other parentage, and so forth, both ancestral, and for progeny.

Meanwhile medical history 126 or the medical history module 126 may begin with birth date, may include all events and exposures within the life of animal that may effect health, as well as any treatments received for actual diseases, inoculations against disease, and so forth.

For example, the breeding schedule and dates, freshing date, gender, and the like may all be included. Meanwhile, vaccination schedules, the attending veterinarian, any prescriptions, and the like may also be included in an animals history. Meanwhile, the events may be as broad as desired to be described. Typically, medical history events may include all diagnosis, inoculations, exposures, and so forth. Likewise, all treatments may include everything from treatments for specific diseases, to generalized vaccinations, tests, pest treatments, and so forth.

The general history module 128 or history module 128 may include any date, with the corresponding location, and event related thereto. For example, certain animals may be inoculated, branded, sold, purchased, shipped, moved between ranges, moved between pens or corrals and the like, may be passed through a milking parlor, may be put into dry herd management, may be located in breeding pens, or the like. Accordingly, any event considered significant may be identified in the history 128 of an animal.

Various key fields, and the links to obtain further data may also be available in the history 128. For example, a particular event, such as a test, may be conducted by a veterinarian, and certain keys or links may link to that event, to that physician, to the function of the event, to the medicaments administered at that event, or the like. All may be included at any desired level of granularity in time, space, population and so forth. Events may include auctions, roundups, milking cycles in a dairy, and so forth.

In general, a record 100 of a livestock animal may be populated with data corresponding to beef production, milk production, calf production, show attendance or winnings, races, and so forth. Race horses, dairy cows, beef cattle, brood cows, beef steers, feeder calves, and the like, all have histories 128. Therefore, history data 128 can be saved in their record 100. Similarly, each has a medical history 126 that may be included in its record 100.

An enterprise record 102 may include the name of the entity, various identifiers (ID), from the actual name of the corporation, to certain governmental and regulatory identifiers. These fields may include a tax payer identifier, registration numbers with various agencies of state, local, and federal governments, ownership information, and anything else that may be desirable to maintain in such a record 102.

Similarly, a facility record 104 may include some identifier, both identifiers exclusively for the database 84 and its operation, as well as other identifiers, including names, addresses, or the like, and so forth. Thus, a facility record 104 may correspond to any facility, and any subset of a facility to the extent that a facility is desired to be identified. A single pen, corral, milking parlor, or the like may be identified. Typically, identifiers may include names, addresses, global positioning system coordinates, database identifiers, any numerical system of identifiers, or the like.

Likewise, a facility record 104 may also include identifications of type as to the function of the facility. For example, some corrals are for sorting, culling, breeding, gestation, and the like. Meanwhile, other pens are for entry into the milking parlor, exiting from the milking parlor, roundup corrals, processing for treatment for disease, de-horning, branding, and so forth. These facility types may vary with the type of livestock maintained. Meanwhile, any other details may also be included in the facility record 104 that may be useful. These may include time dependent information, such as events that occurred at the facility, the dates thereof, and so forth.

Another type of record 90 is a personnel record 106. In general, people have names, various identifying information, both for the database, and as to governmental entities, employers, and the like. Accordingly, various identifiers may be contained in the record 106. Likewise, relationships may be included between a person identified in a personnel record 106, and other locations, operations, businesses, enterprises, animals, and the like.

Typically, an attendant record 108 is a specific type of a record, and may have a key field shared with a personnel record 106. For example, an attendant may be a manager, veterinary staff, including veterinarians, nurses, care staff, and so forth. Meanwhile, cowboys operating in a beef operation, and other personnel administering food, water, medicaments, or other care may be identified as attendants in an attendant record 108. Thus, in a record of animal, the attendant at a particular event, or the list of attendants may be identified. Meanwhile, herd records 110 may include dates, locations, and member identifiers. For example, a herd may be made up for a specific purpose. Typically, in a beef operation, a herd may exist temporarily during a spring or summer roundup for branding new calves. Similarly, a fall roundup may involve identification of certain animals for shipping or culling. Likewise, identification of calves, processing of calves by branding, weighing, or other identification, and the like may occur at a particular event. Thus, a roundup may actually accumulate a group of cattle that will never all be together again.

By contrast, a dairy herd may include herds that exist as milking herds, gestation herds, freshening herds, and the like, isolated from one another for purposes of efficiency in care for the animal at different stages of its productive life. Herd records 110 may be established at any level desired, from the entire population under a single enterprise, to various populations in various pens to various populations in multiple locations having a similar function or some relatedness otherwise. Thus, herd records 110 may benefit from having dates as well as locations identified quite specifically, in order to maintain a detailed record that can be searched later for statistical purposes, alerts, and so forth.

Likewise, geographical records 112 may include maps, global positioning system identifiers (coordinates), layouts from drawings, photographs, or the like. In general, geographical records 112 may correspond to places. In order to identify histories, geographical records 112 may be keyed to correspond to such things as events, animal record dates, and so forth.

The geographical records 112 may constitute a database of geographical information collected over many, many transactions and over many locations. Thus, in general, geographical records 112 may include any identifying information, that may be useful in identifying a place, at a time. Typically, geographical records 112 may also include sources of materials, dates on which material is collected, and so forth. Thus, in tracking herds, animals, and events, one may replicate the geographical information from a geographical record 112 that corresponds to place, date, and content corresponding to the date, place, and other salient information.

Product records 114 typically will include a manufacture, a trade name, a date of manufacture, and so forth. Typically, a product record 114 may include a bar code representation, data that can be retrieved from a bar code or corresponds to a bar code, and other information including chemical names, common names, functions, and so forth. Typically, by scanning in a bar code from a bottle of vaccine, a pill, a package, or the like, an attendant may gather all the useful, valuable, and necessary information related to a product.

For example, at a particular event, animals may all be vaccinated. A single swipe of the bar code may identify the SKU number, from which all the other product information may be accumulated and entered into a product record 114. Thus, product records may become key information when keyed to fields in various other records 90 in the database 84.

Typically, outputs are requested from a database 84 on a regular basis. Accordingly, result modules 116 or result records 116 may include lists of outputs in response to specific queries. Result list 116 may be output and not saved, or may be saved for some time, based on a record retention policy. Typically, entries in a result list 116 may also be filtered, sorted, and otherwise searched as records in and of themselves.

Typically, reports 118 may be output to a specific requester by name, and will typically be identified by a date. Meanwhile, distribution of the report may include the names, titles, affiliations with enterprises, facilities, and so forth of various personnel who receive reports 118, request reports 118, or the like. Meanwhile, the content of the report may be central to the storage of the database 84, as well as certain key words, and identifiers that may make searching easier. For example, any of the records 90 may also have search fields, including key words, identifiers, flags, and the like to render searching more rapid.

Event records 120 may correspond to any activity worthy of note. In some operations, only very few events occur during the life of an animal. For example, in beef herds, roundup periods are infrequent. Often, grazing is on public lands. Public land information may be maintained in geographical records 112. Meanwhile, event records may include events such as roundups, treatment sessions, testing dates and places, with other identifying information, and so forth.

Thus, event records 120 may be keyed with identifiers that can be used in other records as key fields to identify herds in herd records 110 corresponding to certain events identified by event records 120, for example Likewise, any individual animal record 100 may also include many key event identifiers from an event record 120, thus identifying every event identified in an event record 120, that may have affected the animal identified by the animal record 100.

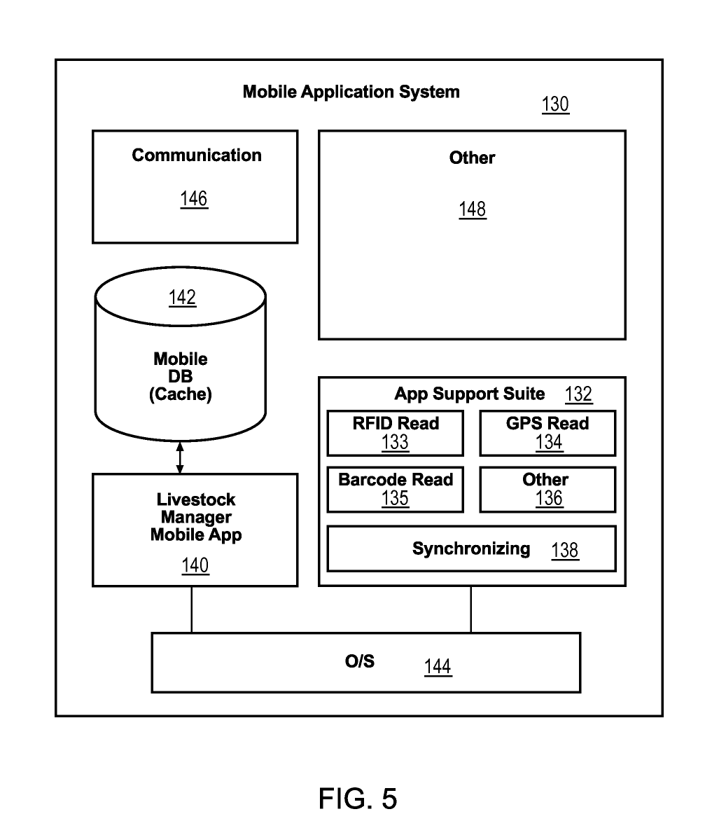

Referring to FIG. 5, a mobile application system 130 may be hosted on a mobile device 80. A mobile device 80 will typically be used in the field by an attendant at an event, affecting various animals. Accordingly, a mobile application system 130 represents the software that will run on a mobile unit 80. Typically, a mobile application system 130 may include an application support suite 132.

The support suite 132 will typically include an RFID module 133 for interfacing with a radio frequency identification reader system corresponding to an event, or the like. Typically, such an RFID reader will be posted at a gate, in a portal, along a chute, or at another location that will be passed by an animal. Accordingly, the RFID module 133 coordinates and interfaces with the information that will be received by a reader from, and therefore pertain to, a particular animal or head of livestock.

Likewise, a GPS module 134 may interface with a GPS system for reading GPS information into the application system 130. Similarly, a bar code module 135 may receive bar code information. Other modules 136 may handle other incoming information, outgoing information and the like.

Meanwhile, a synchronizing module 138 may be responsible to interface with the database server 74, and particularly the synchronization listening service 88 or synchronization listening module 88. Thus, the synchronizing module 138 in a livestock manager mobile application system 130 may be responsible to send the information to be heard by the synchronization listening module 88. Accordingly, the synchronization listening module 88 may pass that information across a link 76 to the OCA module 86 for incorporation into the database 84 by the database server 74.

Typically, the software in the mobile application system 130 may operate on top of an operating system 134 of the mobile device 80. Thus, not only the livestock manager mobile application 140 will exist, and be hosted on the operating system 144 or on the mobile device 80, but other applications 148 may likewise. Meanwhile, many mobile devices 80 may simply be personal devices, mobile telephones, or the like. Many of these will have a communication system 146. Typically, a mobile unit 80 configured as a tablet 80c may include a communication package 146. Likewise, other PDA's, smart phones, and the like, as mobile units 80 will have communication systems 146 or communication modules 146 responsible for communicating voice, data, or both.

The livestock manager mobile application 140 is the application 140 responsible for interfacing with the mobile database 142 or the cache 142 in the mobile application system 130. Typically, in an apparatus and method in accordance with the invention, a mobile database 142 represents a copy taken at a specific time from the database 84 by the database server 74, and delivered to the mobile unit 80 hosting the application system 130. Thus, the mobile database 142 may be managed by the livestock manager mobile application 140 in order to add information, search information, draw necessary information, and create records, fill records, and so forth in the field, and “on-the-fly,” so to speak.

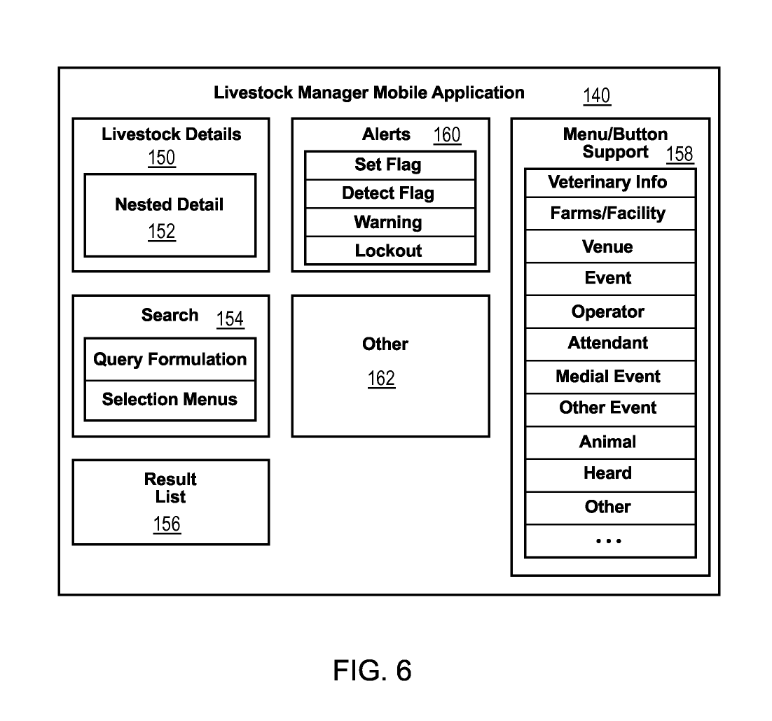

Referring to FIG. 6, the livestock manager mobile application 140, the mobile app 140, may include several executables for operating with the information received from the support suite 132 and the mobile database 142. For example, livestock detail 150 may include any and all information selected by a field attendant relating to any particular animal, herd, group, location, or the like. Various other nested detail 152 may be included therein. Livestock details 150 may include anything in the database 84 that may be useful and tied to the livestock being investigated, treated, or managed in the field. Likewise, a search module 154 may be responsible for query formulation, and may include various selection menus for a user to select query information, of certain standard typest. The search module 154 may provide for rapid searching without extensive interfacing with the user, such as typing. Menus and buttons may support selection of standardized options.

Likewise, a result module 156 or result list module 156 may provide various results, in a format selected by a user, and uploaded, downloaded, or configured by a user on site.

A menu support 158 or menu/button support module 158 may support various menus, selection buttons, and the like. For example, in order to make more simple and readily available the information, decisions, and so forth presented to an attendant associated with a particular mobile unit 80 in the field, information may be available at the click of a button, rather than by queries, text inputs, or even by extensive menus and so forth. Meanwhile, menus may be nested in such a manner as to render the information rapidly available, quickly linked or linkable to other activities, record creation, and the like.

For example, the support module 158 may provide rapid access to veterinary information, farm (e.g. location) information, facility (e.g. function) information, venue, events, operators, attendants, medical events, other events, animal records, herd records, and other records. The support module 158 may allow a user to simply pull up a template, click certain buttons, and have other buttons automatically dimmed, removed, or presented in order to enable the mobile unit 80 in the field to be more user friendly. It may do more of the administrative work, leaving the field attendant available to work with the cattle, horses, sheep, fowl, swine, or other livestock.

A valuable module in the mobile application 140 may be an alerts module 160. The alerts module 160 may be responsible for handling flag traffic. For example, alerts may come in from public, commercial, community, state, national or worldwide health organizations that monitor epidemics, individual diseases, progress of diseases, individual herd infections, outbreaks, and the like.

The alerts module 160 may be responsible to set flags as a result of operations within a herd, or related to an individual animal of livestock. Similarly, the module 160 may be responsible to detect flags incoming from data received over the Internet 72 corresponding to such health organizations listed above, other entities, other operations, other facilities, and the like.

Meanwhile, the module 160 may include certain criteria, settings, algorithms, and the like for generation of or processing or warnings. These may include triggering systems and management systems to receive warnings, process them, and deliver information for decisions related thereto. Similarly, the module 160 may be responsible to formulate, send, and otherwise handle warnings that should be generated at the point of operation of the mobile unit 80.

Meanwhile, lockout information or commands may be received, sent, and the like related to immediate control of emergency situations wherein a particular event should be halted, an animal should be not added, not treated, shunted aside quarantined, and so forth. Thus, the alerts module 160 may vary in its inputs and its outputs from suggestions, to instructions, to actual overriding control asserted by or against the mobile application 140.

Meanwhile, other application modules 162 may also be included, dealing with administration, data collection, training, management, communication, and so forth.

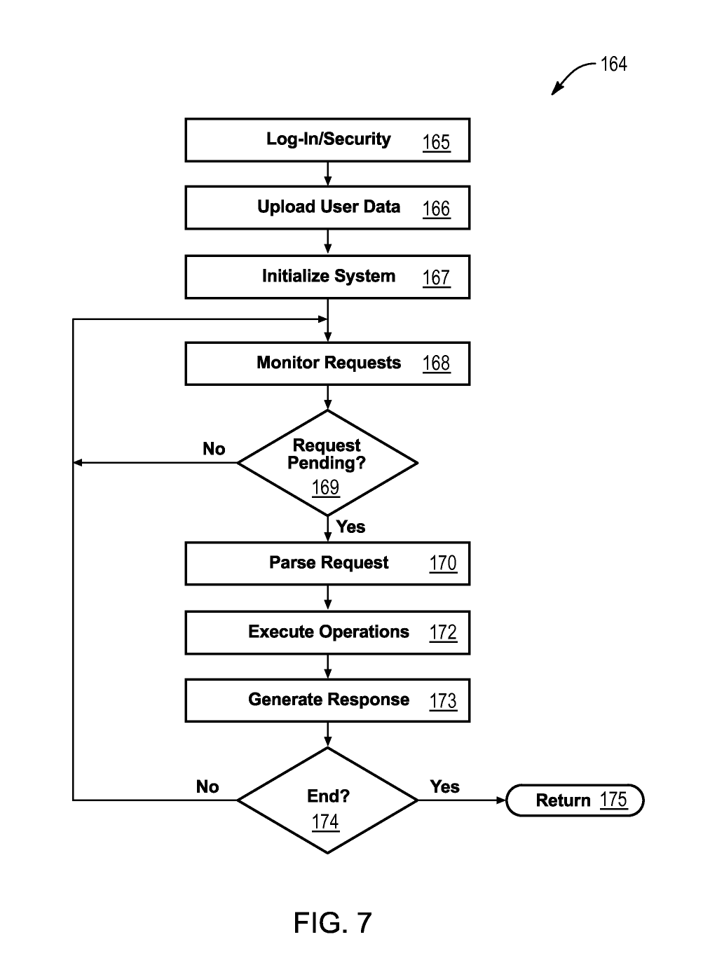

Referring to FIG. 7, a server process 164 may begin with a login 165 or security 165 process. Each of the process steps 165-175 may correspond to a particular module 165-175 corresponding to the execution thereof. Nevertheless, it is sufficiently instructive to discuss the process 164 in terms of the various steps. Thus, logging in 165 may include verification of access, identification of users, identification of affiliations with facilities, entities, enterprises, and any other identification information related to time, space, personnel, control, and the like.

Likewise, login 165 may involve certain keys, codes, and identifiers corresponding to monitoring organizations, reporting organizations, or oversight organizations to be reported to. For example, many states control transport of animals, and must be notified of certain information. Accordingly, the login 165 may require compliance with certain such governmental administrative procedures, and may implement algorithms to do so.

Meanwhile, an uploading step 166 may upload user data, which may be set at any level, and may include many nested levels. For example, user data 166 may be data corresponding to an entire enterprise, to a particular facility, or venue such as a farm, barn, corral, lot, pen, building, or the like, and an individual attendant in charge, an attendant actually dealing with an animal, or the like.

Uploading 166 may be controlled by various parameters in order to provide an upload of the information from the database 84 according to the information needed. Likewise, initializing 167 may involve obtaining information from the database 84 required to set up a particular population, herd, animal, operation, event, or the like.

Monitoring 168 requests may be done substantially continually while the server 164 is operational. This may operate on a full time basis, day and night, around the clock, day in and day out, throughout the year. Polling or interrupt driven monitoring may be used ay any time. Typically, a test 169 determines whether a particular request is pending from a user. If not, then monitoring 168 continues.

On the other hand, if a request is pending, then the test 169 advances the process 164 to parsing 170. Parsing 170 the request may involve various manipulations compariosn, analyses, and so forth of character strings, unicode, numbers, and other inputs. Following parsing 170, executing 172 the operations requested may occur. Typically, the database search engine 94 may be responsible for parsing 170. Meanwhile, the search engine 94 is also typically responsible for executing 174 the search and returning a result.

Usually, a particular user who has made a request may also have some input through the user interface 92. For example, the query template in a user interface 92 may control the uploading of a request, and a downloading of the result. Accordingly, generating 173 a response may involve outputting by the search engine 94 information searched and downloaded from various records 90. Meanwhile, the user interface 92 may format that information in accordance with a format previously determined by the user or requestor.

Ultimately, if the test 174 indicates that the request has been fully complied with, then the system may return 175 to its previous condition. If the request has not been fully complied with, and other records need to be drawn up from the database 84 for responding 173, then the test 174 returns the process 164 to monitoring 168, as illustrated.

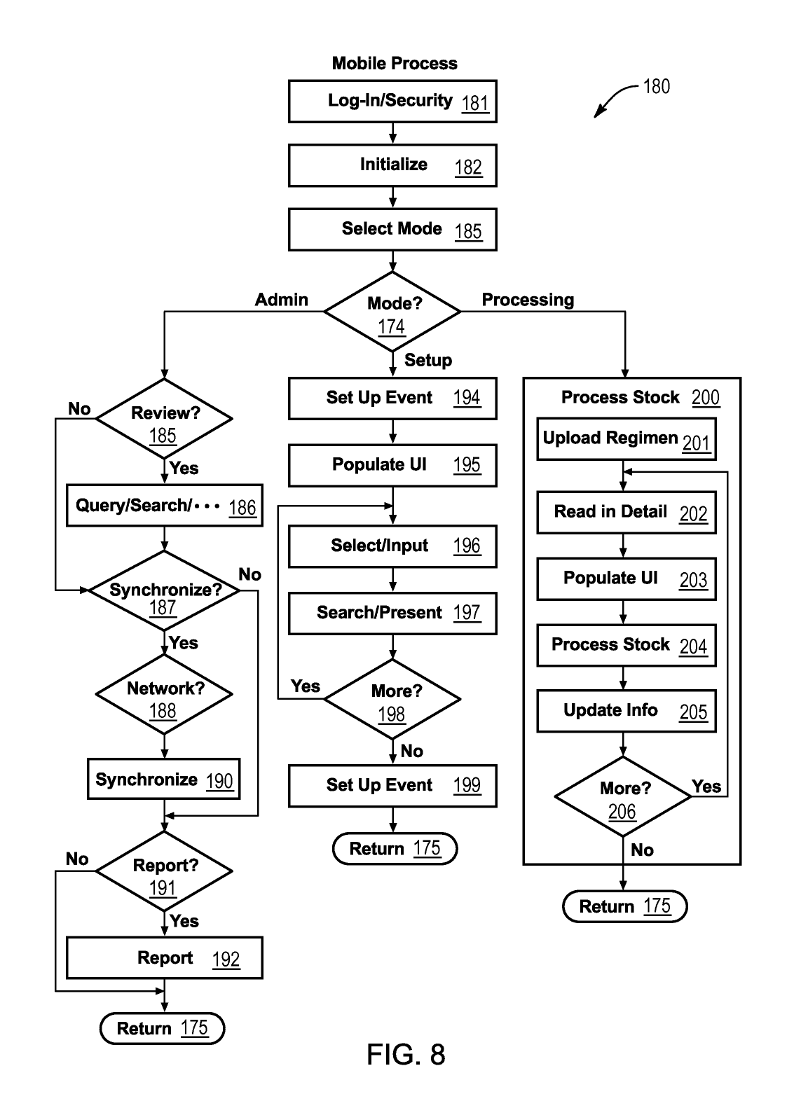

Referring to FIG. 8, a mobile process 180 may include a login 181 or security 181 step followed by initializing 182 as described hereinabove. Typically, these processes will occur with a mobile unit 80 and a single user interacting with that mobile unit 80. Meanwhile, selecting 183 a mode may be followed by a test 184 directing the process 180 between various modes (e.g, review, synchronizing, reporting, setup, processing, etc.). For example, initially, a review mode may include a test 185 determining if records are to be reviewed. If so, then a query 186 may be posed, with a subsequent search, and reporting back.

Thereafter, a determination may be made whether synchronizing is to be done. A test 187 determines whether synchronizing is needed. If synchronizing is requested, then a test 188 determines whether a network is available. Often the mobile units 80 are very distant from access to the Internet, and not connected to a network. This is partly a matter of cost, partly a matter of geography, and partly a matter of convenience, to say nothing of the lack of coverage and the possibility that this lack may trump every other consideration. Ranches may be far flung from the areas of cell phone towers or other communication links.

If synchronizing has been determined, and a network is available, then synchronizing 190 may occur. Typically, reporting may be requested. Accordingly, a test 191 determines if a report is requested. If so, then a report 192 may be generated or reporting 192 may occur. Again, as with FIG. 7, the process 180 is made up of numerous steps. However, each of the steps illustrated may have or represent a corresponding module in software to execute the respective functionality. Following reporting 192, the system may return 175 to its previous condition.

If the process 180 is not to move forward at any of the steps 185-192, then it may return to the appropriate intermediate step thereabove and continue. Ultimately, however, the system will return 175 in order that another mode may be set, determined, or otherwise selected, and thereby be tested by the mode test 184.

In a setup mode, a setup 194 may prepare the administrative information for an event. Accordingly, the process 180 will populate 195 a user interface in order to provide the data to an attendant who needs to input that information. For example, at a roundup, branding, test, or treatment event the user may need to populate the user interface with the functions, the persons, and the treatments that will be administered.

Meanwhile, selecting 196 or inputting 196 certain data will control the operation of the event, and the treatment of each of the livestock animals. Thus, a search 197 and presentation 197 will also provide information, from templates to data to instructions to the user in response to the selections 196, inputs, and the like.

If more information is to be input, a test 198 will return the process 180 to continuing to select an input 196, as well as searching and presenting 197. Ultimately, when no more is to be done, no more decisions are to be made, or information reviewed, then the process 180 proceeds to save 199 the regimen or protocol that will be associated with the event planned. Ultimately, the process 180 may then return 175 to the mode test 184 or other location, according to the dictates of the user.

A third branch of the process 180 is the process branch, or mode. Accordingly, processing 200 stock involves the actual event, or operation of certain attendants at an event, at a location, involving one or more head of livestock. Again, the process 180 uploads 201 a regimen, which has been saved 199 from previous planning. Planning and setup may have occurred days ago, weeks ago, months ago, years ago, or minutes before.

Meanwhile, reading 202 may read in the details from an animal radio frequency identification tag Likewise, bar codes, global positioning systems, and the like may be read 202 in order to populate 203 the user interface at the actual event. This information may be populated early, late, one animal at a time, or as a group.

For example, if the entire group passes through a chute, gate, portal, or the like into a holding pen, then many of the details corresponding to each animal may be found in the database after an identifying tag is read 202 at that time. Populating 203 the user interface may occur in real time or before any actual processing 204 of stock (livestock) occurs. Thus, in due course, according to the particular identification associated with an animal, and the protocol determined by the regimen saved 109, the processing tool for livestock may continue until completed. Ultimately, information may be updated 205 on an individual head basis, and on a population group, or herd basis according to the grouping that is affected by the processing 204.

In the illustrated embodiment, a test 206 may determine whether more processing 204 is to occur. If so, then the process 180 may return to reading 202 the details for another animal, populating 203 the user interface accordingly, and moving forward with processing 204.

On the other hand, if no more is to be done, with the group, or with the regimen, then the test 206 may return 175 the processing of stock to the test 184, which may ultimately return to exit the process 180 entirely.

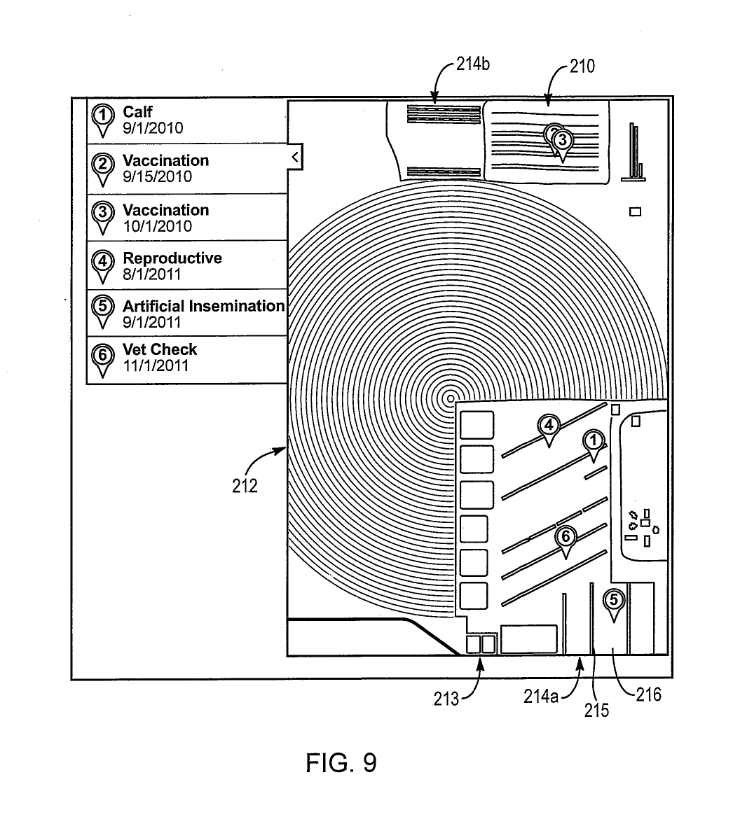

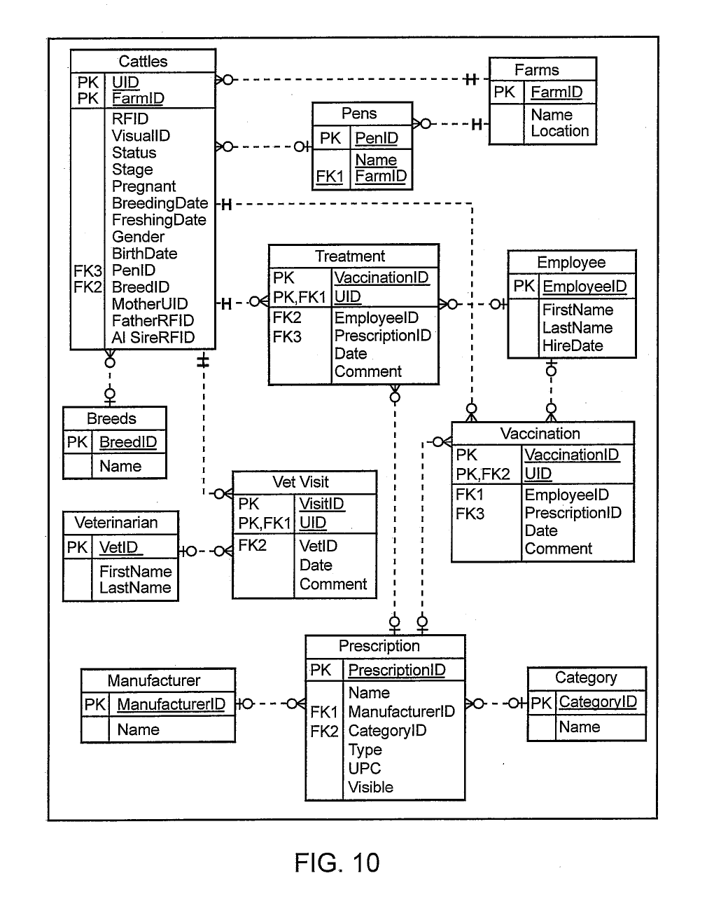

Referring to FIGS. 9 and 10, a system for integrating information in a visual format may include providing a visual integration presentation 210. For example, the visual presentation may include an actual image, either by schematic, or by actual photographs. The visually integrated presentation 210 may illustrate production fields 212, as well as feed stores 213. Likewise, operational areas 214, such as corrals, pastures or pens, typically as living space 214a, processing space 214b, treatment space 214c and the like are visible. Thus, the various operational areas 214 may be identified visually.

Operational areas 214 may include sheds 215, open spaces 216, and so forth. Meanwhile, various regions may be divided by fences 217, gates, portals, RFID locations, GPS coordinate positions, and the like. Thus, an individual may quickly see where the particular functions, regions and material, as well as animal herds and individual animals, may be located, were previously held, were previously treated, and so forth.

In the illustrated embodiment, various event locations 218 may also be identified. In this case, six events are identified by location, corresponding to a single animal, these events involve a calving location 1, a vaccination location 2, a vaccination location 3, a reproductive region 4 or holding pen for reproductive maintenance, and artificial insemination holding area, a veterinary check region 6, and so forth. Any data desired may be configured to be illustrated on a visually integrated presentation 210 in accordance with the invention, in order to render much more understandable and much more quickly so, to a user, the information presented by a mobile unit 80.

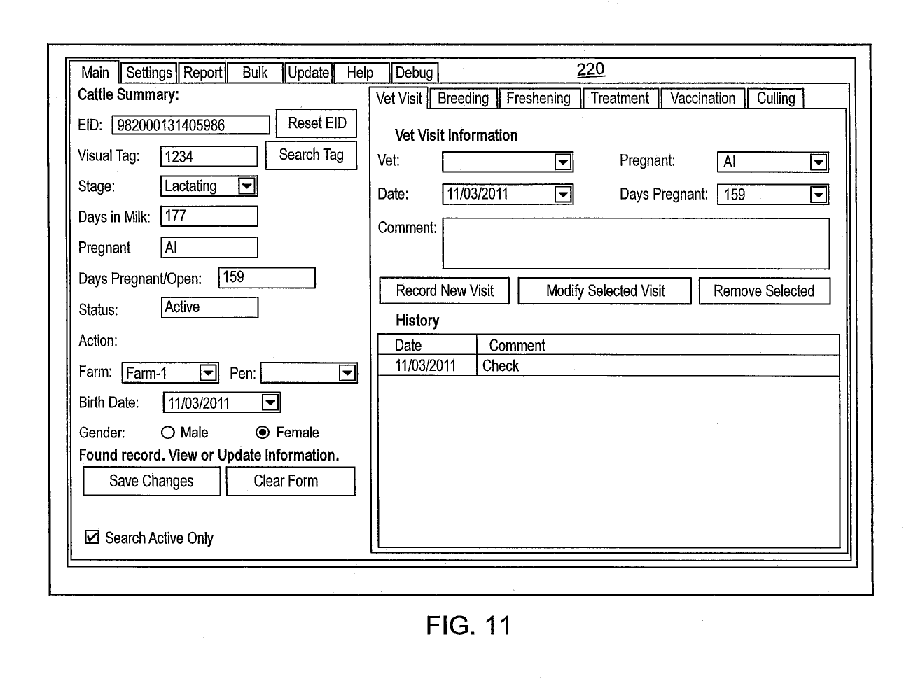

Referring to FIG. 11, a screen shot of a template 220 corresponding to a particular animal summary is illustrated. Various templates 220 may be configured to provide any and all information available from the database. Similarly, templates 220 may be configured for receiving inputs, providing outputs, reporting in standard formats, receiving and responding to queries, and the like Likewise, templates 220 may be configured for managing queries, in order to guide a user interactively by changing the available input fields for a query, once certain other fields have been selected, and inputs have been provided thereto.

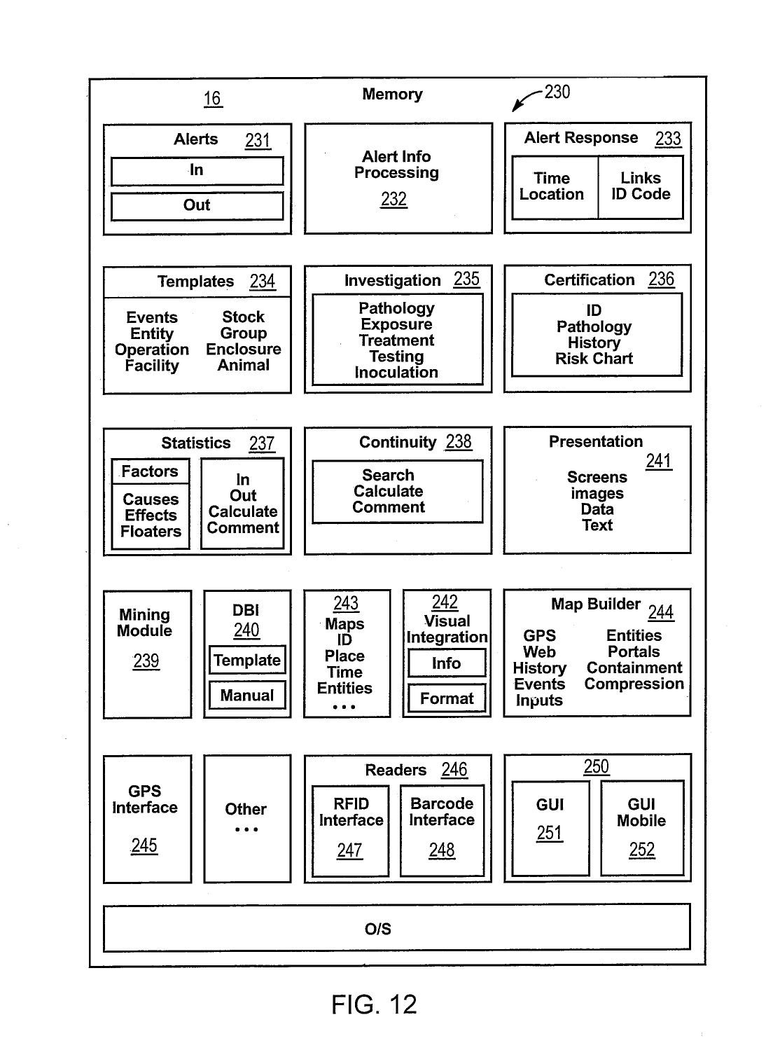

Referring to FIG. 12, a livestock certification system 230 is illustrated. In the illustrated embodiment, the system 230 may be configured as software running on the server 74, a server 78, or the mobile unit 80. As a practical matter, the system 230 may be distributed across several units, and may terminate at a node 12 corresponding to a desktop unit 82 at a headquarters of a particular operation, facility, or the like.

Referring to FIG. 12, a system 230 for certification of livestock may include a variety of modules, each of which may represent a functional step in a process for certifying livestock against various diseases, exposures, populations, genetic weakness, and so forth. In the illustrated embodiment, the livestock certification system 230 may include an alerts module 231 for managing incoming alerts, receiving, processing, and otherwise disseminating the information for compliance therewith, and so forth. Likewise, the alerts module 231 may include an outgoing alerts module for generating outgoing alerts relating to livestock under control, or within the purview of the system 230.

A processing module 232 for alerts may include elements for handling incoming and outgoing alert management. For example, an alert may concern the discovery of a communicable or highly infectious disease outbreak within an animal, a herd, a locale, population, and so forth. Similarly, such a system may actually pertain to a slaughter house for beef cattle, the production of milk in a dairy, a disease outbreak in a veal operation, and epidemic in a sheep operation, or the like. Accordingly, the processing module 232 processes the information, statistics, and data to determine the presence of a risk, the probability of that risk, and that dissemination of information regarding that risk, as well as a collection of additional information available to track, trace, broadcast, ameliorate, quantify, or communicate risk information.

In addition, an alert response module 233 or response module 233 may be responsible to formulate and prepare alert responses. For example, times, locations, identifications, connections, and the like associated with livestock, whether individual animals or groups, may need to be identified, treated, quarantined, protected, moved, not moved, and so forth. Accordingly, the response module 233 develops the information which will then be used to determine activities associated with the alert, given its content, severity, extent, and so forth.

In the illustrated embodiment, templates 234 may exist for identifying information, and presenting it, both in input format and in output format, for various situations, conditions, facts, and so forth. For example, event information, entity information, livestock information, and the like may all be collected, and displayed, in preselected templates prepared by the users, prepared by a central location for dissemination, or standardized by a particular enterprise, facility, individual, owner, or the like.

For example, information regarding a particular operation, a facility, an enclosure, a group, an individual animal, a particular population, or the like may be collected in a standardized format, to ensure that all information necessary for processing is collected. Similarly, information may be provided as an output from the database 84 through templates 234 in order to assure that a standardized format makes all information presentable and presented in a manner easily understood and not requiring extensive detailed reading or searching within a presentation of text in order to find the key operative information. The investigation module 235 may be responsible for processing an investigation within the data of a database 84 and other inter-related databases.

For example, in one embodiment, the system 10 may actually extend across numerous enterprises, throughout a nation, throughout a state, or even across the world, and the like. Accordingly, an investigation module 235 may mine the data in the database 84, and other related databases. For example, the investigation module may have other submodules responsible for searching, identifying, listing, and otherwise providing pathologies. Pathologies may include diseases, organisms, certain conditions, various pathogens, whether chemical, biological, or the like, and so forth.

Similarly, an investigation module 235 may have an exposure submodule responsible for tracking, linking, and otherwise determining exposure of animals or groups of animals, such as herd populations to a particular pathology or carrier thereof. Meanwhile, treatments, testing, inoculations, and the like may be the subject of the investigation module 235 in searching for key information to identify difficulties and resolve risks.

A certification module may provide identification, pathologies, risks, typically charted in terms of a particular risk, and a particular percentage of probability of occurrence of a particular pathology. Similarly, risk charts may also provide such information as the exposure or injury to animals, and thus a corresponding probability of decreased productivity. For example, milk production, health, longevity, and other animal health factors may be identifiable by animal, by population, or the like.

A statistics module 237 may have responsibility for identifying factors, such as causes, effects, and floating factors. For example, floating factors may be those that are simply items to be observed, and possibly correlated, without necessarily knowing whether they are causes, effects, or the like. Floating factors may later be determined to be causes or effects, but may be tracked, simply as a bookkeeping or statistical exercise, since they can be known, and may correlate with other factors. Thus, causes, for example, may be thought of as independent variables, while effects are dependent variables having a functional relationship and dependents upon causes. Floating factors need not have an identifiable role as a cause or an effect, and may be the subject of research or investigations.

Meanwhile, the statistics module may provide for inputs, calculations, correlations, and outputs relating factors to one another in a suitable, and typically a graphical, format readily understandable and interpretable by a user.

A continuity module or a continuity linking module 238 may provide for searching, calculations, and comments regarding additional links identified by a search, or an investigation of a particular animal or population. For example, within a few generations, a genealogy of an animal grows to include many animals, and links to many populations or herds.

Likewise, a genetic defect discovered with respect to a particular animal may result in an influence in an exceedingly high number of progeny who may be effected. Accordingly, genetic defects studied by population may be difficult to process or disseminate across an entire possible suspect population. Accordingly, a continuity and linking module 238 may provide for a background linking and identification of other groups, which may be studied as a result of discoveries or influences identified with respect to a particular animal or a particular population. Meanwhile, the statistics module 237 may process risk rates. These may be calculated in order to limit or bound at some point the continuity and linking module 238 in its linking of proximity of one animal or population to another.

A mining module 239 may mine the database, Internet websites, news services and other databases, in order to continue to update information that may be useful in management of an animal population or livestock herd. The mining module 239 may be responsible to search for any parameter with respect to any population or animal, including studies and information that may be published in papers, books, articles and the like.

A database information module 240 may include a set of templates, menus, and the like for use by the database in collecting information. A template may identify types of information to be obtained, criteria for passing or recognition, and the like. In a template, fields may exist for receiving text, numbers, binary large objects, images, strings, files, or the like.

A presentation module 241 may provide screen templates for presenting information. For example, images, data, text, and the like may be presented in a particular mode, and the presentation module 241 may be responsible for such formatting in order to provide a consistent appearance and ready access, as well as easily understandable information. As part of the presentation module 241 or independently therefrom, a visual integration module 242 may provide informations, formats, and the like to integrate information, in order to provide a better understanding thereof, and a more understandable presentation thereof.

Graphical user interfaces provide for visualization on a scale heretofore unknown. Accordingly, a visual integration module 242 may be responsible for superimposing, embedding, and relating information from different sources, onto a single map of suitable domain, such as time, space, or other variable, onto a single record or presentation relating to a particular animal or population.

In certain embodiments, a map builder 244 may involve mapping an identification of the specific location and space, typically bound to a particular time period. For example, when an animal or population of animals is passed through certain portals or is held in certain containment units (pens, corrals, facilities, etc.) those items have specific locations and boundaries. Likewise, over histories of locations, they may change their boundaries. Accordingly, history maps may identify a location, and a configuration of containment units at points in time, over a period of time, and so forth.

Similarly, event maps may identify the locations in space, on the surface of the earth wherein events happen. These may be maintained in any specific system of identification as may be useful. Accordingly, GPS coordinates that are now within feet of accuracy may identify the locations of such events. Meanwhile, the map builder 244 may include submodules for GPS management, web map management and presentation, inputs of maps, mapping of entities, and the like.

Meanwhile, a compression system may identify selected words, codes, and otherwise process information in order to reduce the total amount of data necessary to store the information needed. For example, much data may be consolidated by the compression system by bounding the number of portals, the number of access points through which animals may pass in and out of a holding system, by bounding holding systems more precisely, and so forth.

A GPS interface 245 may operate to process GPS information into and out of the system 230. An RFID interface 247, a bar code interface 248, and the like may be part of livestock identification 246. That is, regardless of the mechanism by which livestock may be identified, from brand location, to brand shape, to RFID number, bar code, ear tags, and the like, the animal identification interface 246 may be responsible to process that information, determine protocols, and maintain that information as it is transferred into and out of databases throughout an operation, throughout a facility, throughout a geography or jurisdiction, or throughout the world.

Likewise, a user interface 250 may include a graphical user interface 251a graphical user mobile 252 peculiar to mobile units 80. This may typically use a reduced set of instructions, reduced space, and so forth in order to render the proper information available, taking less time, less processing, and less resolution such as may be required by a display screen that is smaller.

A system 70 in accordance with the invention may be configured to operate under software as a service (SaaS). Advantages and disadvantages of the SaaS model have been discussed in literature, and may benefit from developments in cloud computing and emerging Web 2.0 technologies. Periodic or large scale data storage, processing capacity and accessibility may provide back end services for mobile applications. With occasionally connected applications (OCA) in conjunction with a web based service, field workers may access the data indirectly through a cache stored locally on the mobile devices.

The application 130 will connect to a database server 74 periodically and synchronize 190 its content with the local cache 142 as needed. While it still requires a wireless or cellular network connection for operation, because of its non-real-time nature, limited connectivity at key locations can sufficiently support the occasional connection to update information.

In one embodiment of an example system, The Microsoft SQL Sync Framework provided a mechanism for an OCA to easily maintain and update data between mobile devices 80 and a database server 74 through compact and efficient exchange protocols.

The application 140 (system 130) is no longer tied to a fixed workstation 82 in a single location. Farm managers access information and make decisions through any web enabled devices 80 (PC, laptop, Mac, tablet . . . ) at any location with an Internet connection. This is particularly helpful for farmers will multiple farms in difference locations.

Data synchronization 190 occurs directly between the field mobile device 80 and the remotely database server 74. As soon as the device 80 is in range for wireless Internet access, a user may update data with one click in the mobile application 130 on the mobile device 80. The latest data base 84 information is immediately available on the web service 78. Multiple mobile devices 80 operating in the same farm systems may also synchronize 190 with each other using the same mechanism without having to do it in the central office. Data are automatically replicated and backed up on daily basis for disaster recovery as needed.

The Microsoft SQL Server 74 supports Unicode character strings in the database 84, so the client 130 and server 84 software may be localized for other languages. This may be important, especially for developing countries, which lack sophisticated software systems for farm and animal management.

It is contemplated that this web application 130 may integrate with other web services available. For example, the location information collected through the GPS enabled mobile devices 80, when overlaid with a mapping service such as MapQuest, Bing Map, or Google Map provides a visual context for an animal's current location and history.

It is contemplated that the system may grow in its connections to higher levels of inclusion. For example, as a host system collects more animal data from multiple farms in multiple locations it may cross any boundary of ownership, association, geography, or jurisdiction desired. The system has the capacity to analyze data statewide, nationwide, and worldwide to discover knowledge, extract predictive information to improve herd health and increase production, and provide alerts and searches to quarantine diseases and genetic defects.

The prototype system was developed with Microsoft Windows-based mobile devices 80, RFID readers 133 in the field, and Microsoft Windows Azure cloud services as the server platform 74 for the web applications 78, 88 and database 84.

The main components included a central data source 84 where all the farm and animal information were are stored. A web server 78 running the applications 164, 230 (although not all modules thereof) remained accessible from any web browser. It provided a desktop-application-like interface to view, manage, and update the information stored in the data source 84.

The initial server software 78 was developed on the Microsoft Azure Cloud using a combination of Web 2.0 technology such as C++ and JavaScript programming languages to provide dynamic and interactive content through the web browsers. Mobile clients 130 ran on Microsoft Windows and Windows Mobile operating systems. The client 130 or mobile unit 80 relied on a graphical user interface 252 (GUI) application and a local SQL database 142. The GUI application 252 interfaced with peripherals such as an RFID reader, barcode reader, and a GPS. The mobile unit's local database 142 worked as a cache 142 of the central data source 74, 84, which synchronized 190 with each other occasionally as needed. The client applications 140 were developed with the C++ and .NET software library.

While a real-time mobile client application 140 would deliver the most up-to-date information and maximum manageability, the cost of network infrastructure and the constraint of battery life usually prohibit such a system. An occasionally connected architecture without these drawbacks offered a mechanism to provide the most up-to-date information in a locally cached database 142 on the mobile client devices 80. This reduced the amount of network traffic exchanged between clients 80 and server 78. Thus, only recent changes since the previous synchronization ever needed to be transmitted.

With a fully replicated database 142 on the mobile client device 80, the mobile application 140 supported nearly full functionality previously only available back at the office desktop software in many current systems. For example, enrolling a new animal, such as a dairy cow, in the field was possible without the farmer having to first create an entry of a new cow on the desktop.

The mobile client application had an interface 297 with the local RFID reader to retrieve the unique identification (ID) number from each animal's ear tag. When the ID number had not been recorded, the associated animal was immediately enrolled in the field. Since the client system 130 included a complete copy 142 of the database 84, information of the newly enrolled animal was populated to the central database 84 during the next synchronization 190 process.

When the ID number retrieved from the reader is already in the database, comprehensive information for the associated animal is accessed from the local database (cache) to be viewed or modified in the mobile client application. Any changes made on the mobile database 142 are synchronized with the central database 84 at the next occasion when network connectivity is available.

A dynamic and interactive web application 140 mimics the desktop application 164 with the same user interface 250 such as tabs, buttons, text box, drop down selection, calendar, and so forth. A user may find a cow or heifer by identification number. A report module 118 creates lists of cows and heifers based on status, conditions, date range and other properties, and creates reports. Reports can be emailed at any suitable periodicity to selected staff as PDF documents.

A user may access and manage farm information such as tracking a veterinarian, workers, medication, vaccination, treatments, observations, and other activities. All users log in 165 to the web application 164 through a common portal. However, each client has its own individual farm database 84 on the server 74. Based on the logged in user's identity, a new session is initiated, connecting to the designated farm database 84 associated with the client account. Multiple user profiles may be created for each client with various levels of access control privileges based on their roles on the farm.

Each client's farm database 84 contains tables 90 of Records 90 storing information on the cows, health and medical treatments, reproductive status, farm staff, and more. The mobile application 140 also connects to the servers 78, 86 with proper user credentials and then to the associated farm database 84. A local copy 142 of the farm database 84 on the mobile device 80 synchronizes 190 with the corresponding database on the server 74 occasionally as needed and as network connectivity is available.

With a GPS connection 245, or a GPS-enabled mobile device 80, the client application 140 associates geographic location coordinates with events 120. New information is added to a record 90 of an animal, recording, for example medication, vaccination or insemination, anytime the status of an animal is changed, (e.g., pregnancy or milk production) or the animal is moved from one location to another (e.g., different farm location or sold).

While this event-based geographical information does not provide real-time location tracking, it provides a history of where the animal has been and in what populations, which information is sometimes more important than the real-time tracking. This event-based tracking does not incur capital infrastructure overhead for addition equipment, but at the same time provides monitoring important to identify, track, and contain any communicable disease outbreak.

As all the geographical data are stored on the server 78, the system 70 (e.g., map builder 244) integrates with third-party mapping services such as MapQuest, Google Maps, and Bing Map. The user display overlays the animal's location history on a map for better visualization 210.

The web server 78 is also used as a vehicle to deliver software updates to the mobile client application 140, which periodically checks 188 to determine if and when network access is available. Users are notified of available software patches and can choose to download and install them through a single click of a button.

The initial prototype was deployed and tested for over six months on a three-thousand-cow dairy farm in central Washington State, USA. The farm operators ran the mobile client application 140 in the field on a rugged tablet PC with Microsoft Windows operating system and SQL Express 2008 R2 database. The web application 78,164,230 and central database 74, 84 were deployed through Microsoft Windows Azure cloud services. Existing wireless network access points were available in various barn, pen, and office locations on the farm. Operators modified data in the field and sent the updates to the servers 78, 88, 86 upon arrival back at locations with wireless network access. The synchronization process 190 was a simple one click operation to send or retrieve data modified since the previous synchronization 190.

The farm manager used the web application 140 to monitor herd status through a browser. The benefit of being able to manage farm information from anywhere instead of tied to a single office location was very well received. As most large scale dairy farms are 24×7 operations, the service oriented software system 70 provided much needed flexibility for the dairy farmers. The web interface also allowed users to configure reports 118 to be generated automatically as PDF documents and delivered to farm managers daily, weekly, monthly and on demand, as email attachments.

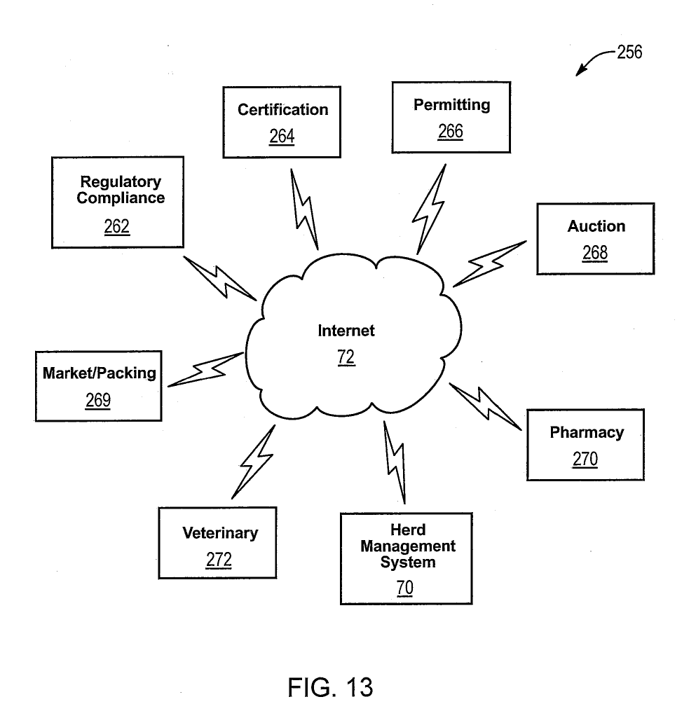

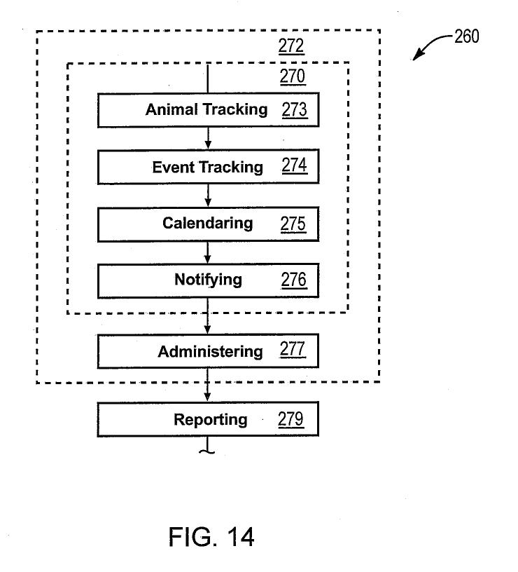

Referring to FIGS. 13 and 14, while continuing to refer generally to FIGS. 1-16, in on embodiment of an apparatus and method in accordance with the invention, a herd management system 70 may interface over the internet 72 or another network or internetwork 72 in order to effect a system 256 of individual software modules 262-272 executing their various processes 262-272 on one or more processors 14. For example, each module 262-272 may also be considered to represent a process 262-272 corresponding to that particular module 262-272, respectively.

In the illustrated embodiment, a regulatory compliance module 262 may be responsible for tracking, evaluating, researching, analyzing, predicting outcomes of, reminding, and recording compliance with, criteria established by regulation. For example, animal transport, particularly across state lines, is a highly regulated activity. Similarly, verification of certain treatments and freedom from certain pathogens (diseases, organisms, etc.) must be verified and documented before animals may be transported, disposed of, bought, or sold.

Meanwhile, such pedestrian verifications as branding, brand inspection and, integrity, bills of sale, and the like may also be subject to regulatory compliance. Thus, a regulatory compliance module 262 is responsible for tracking, evaluating, or analyzing, according to certain established criteria specified, the compliance of an animal, a population, or the like with regulatory requirements. Similarly, the regulatory compliance module 262 may be responsible for predicting, reminding, and recording with respect to compliance with any criterion or set of criteria established by regulation, and incumbent upon a keeper of animals in order to move those animals in commerce. What may be applied to an animal herein also applies to a venue, a livestock operation, a farm, a ranch, a person, a business, a processing plant, or the like. Each is subject to regulation where compliance must be verified.

Similarly, a certification module 264 may be thought of as a software module executable to be responsible for tracking, evaluating and analyzing, according to criteria, predicting results, reminding of actions to be taken, and recording compliance with warnings and reminders as well as those criteria. Thus, a certification module 264 may be thought of as a generalized process, and may be specific to a specific regulation, an industry standard, a protocol of a particular owner or care giver, a regimen recommended or required by a veterinarian, or the like. Similarly, associations, purchase orders from commercial entities, or the like may require certifications or verifications that are implemented by the certification module 264.

Similarly, permitting 266 is responsible for data collection and analysis for creation, verification, and tracking of permits. This may include evaluating records against specific criteria, analyzing data, predicting results in certain circumstances, providing checklists, and outputting reminders based on evaluations of criteria, in order to effect the creation, distribution, and control of permits required.

Again, certain permits may be required as part of any scheme of regulatory compliance from construction to transport to grading of meats. Accordingly, various aspects of the regulatory compliance module 262, the certification module 264 and the permitting module 266 may be held in common, or may be embedded within one another.

Nevertheless, regulatory compliance 262 may require certification by animal, by population, by event, by caregiver, by veterinarian, by other personnel name, by medicament or supplement administered, or some other event or interaction with an animal. Typically, regulatory compliance is an issue of state or federal law. Permitting, effected by the permitting module 266 is typically a state law or even a county ordinance compliance issue. Typically, the permitting module is responsible to generate paper permits, or maintain permitting records that can be accessed, analyzed, searched, updated, transferred, and otherwise distributed in association with actions taken with respect to livestock, especially cattle, including movement, marketing, disposition, identification, inspection, treatment, and so forth.

Ultimately, certification 264 may be any certification 264 in accordance with established regulatory criteria requirements. Criteria may come from permitting, or may be controlled simply by an industry standard adopted, association standard, purchaser specification, purchase order requirement, owner protocol, or the like.

In one embodiment of an apparatus and method in accordance with the invention, a certification module 264 may be responsible for providing certification against certain risks, including exposure to pathogens (e.g., microbes, bacteria, viruses, diseases), verification of quarantine times and effectiveness, verification of contacts upstream and downstream with respect to a particular animal at a particular venue at a particular time, and so forth.

In certain embodiments, an auction module 268 may be responsible for tracking a particular animal or population, and typically both, evaluating that animal or population in accordance with certain desired or imposed criteria, outputting as a result any warnings or calculations of risks. Issues such as predictions of problems, warnings of exposure, or the like result from evaluating the record of the animal or population scrutinized, analyzed, and reported.

Similarly, an auction module 268 may be executed on a computer of a commercial auction, or of an owner of certain populations of animals. Typically, a commercial auction may operate an auction module 268 in order to verify to buyers the histories an freedom from pathologies (e.g., illness, health problems, defects, injuries, exposures, etc.) of animals. Accordingly, sellers who comply with the tracking, evaluating, established criteria, predicted results, reminders of action, and compliance with such actions, duly recorded, may obtain better prices for their animals or their animal products by virtue of satisfaction of such criteria and subjection to such tracking, analysis, and risk assessments.

An auction module 268 may be operated by a commercial livestock auction, but auctions may be operated by various commercial entities for various commercial purposes. In some instances, an auction may be auctioning animal products from a commodities trading venue trading commodity prices according to indices in a global market. An individual auction selling individual animals may operate with livestock online with only identifiers or records. A commercial enterprise may sell animal products, future predicted output of products, or actual animals present at the venue.

One market entity of significance is a packing plant. In general, a market module 269 or packing module 269 may be executed on a computer of a buyer, a seller, a broker, or another commercial entity operating within a market for animals or animal products. Animal products may include, for example, live animals, milk, eggs, fur, hides, leather, meat, and so forth. Thus, a market module 269 may be responsible for tracking, evaluating according to certain criteria, predicting a result or rating for particular products, providing reminders of actions to be taken such as administration of certain protocols, and outputting recommended protocols, as well as recording compliance with those protocols.

Thus, whether specifying protocols for slaughter and inspection in a meat packing plant, weigh and rating wool sheared from an animal or a population of sheep, or managing the handling of any other animal product, the market module 269 may track records, evaluate products, predict problems, remind of treatments, direct treatments, and record compliance with private, association, governmental, or other specifications. Thus, in general, the modules 262, 264, 266, 268, 269 represent part of a system 256 and a process 260 for establishing, documenting, analyzing, judging, rating, pricing, directing, or guiding through instructions and protocols, the compliance with criteria established privately, commercially, individually, or governmentally for controlling, evaluating, and verifying the quality, health, physical characteristics, or other parameters associated with live animals, animal products, meats, fibers, by products, and so forth.

In one embodiment of an apparatus and method in accordance with the invention, a pharmacy module 270 may be responsible to track, evaluate against established criteria, predict a result and schedule, provide reminders, and record compliance by personnel, animals, populations, venues, and the like with respect to pharmaceutical protocols.

For example, animals typically will require administration periodically of medicaments, preventives, quarantines, and so forth. Medicaments may be inoculations administered to animals, pills, ointments, or the like. They may be other treatments, ranging from exercise or minor administration of topical preparations to attended delivery, to preparations or devices used in surgeries.

Accordingly, a pharmacy module 270 may be responsible to track events, locations, populations, personnel, animals, quarantines, times in, times out, durations, and so forth. Likewise, the pharmacy module 270 may be responsible to predict or otherwise analyze the time periods between administrations of medicaments, preventives, quarantines, and so forth. Thus, the pharmacy module 270 may rely on an analysis of data corresponding to an animal record in order to predict and recommend a time for reapplication of a pharmaceutical preparation or an over-the-counter treatment.

For example, upon exposure of an animal or population (herd, group) to a pathogen, a quarantine period may be recommended. Such recommendations or requirements may result from analysis of contacts by the animal with other animals, contacts with potential pathogens, incubation times of pathogens, virulence, contagion periods, and so forth. Likewise some may be almost routine, thus, the pharmacy module 270 may be responsible to search and evaluate records of animals, operations, and remedies to provide analysis and recommendations delivered to an owner, caretaker, or veterinarian responsible to administer such materials, processor protocols to an individual animal or population.

In certain embodiments, the pharmacy module 270 may rely on the database 84 of the herd management system 70 in order to effect its analysis, notifications, and so forth. As a practical matter, medicaments and preventives need not be isolated by a bright line division. Nevertheless, in general, a medicament may be thought of as a drug or remedy applied to treat a specific pathological condition, such as an illness, known exposure, or the like. In contrast, preventives may typically be applied regularly in order to prevent certain pathologies.

For example, animal populations are routinely sprayed for removal external parasites, treated with chemicals for killing internal parasites, and dipped or sprayed for insect larve, such as horseflies, bot flies, and so forth. Typically, such treatments may be applied upon detection of such pathogens. However, they may also be applied periodically in order to keep such pathogens at bay, regardless of whether an inspection or detection of such pathogens was actually done.

Similarly, quarantines may be recommended based on dates of potential or actual exposures. They may also be recommended based on detections made by veterinarians or others of actual pathological conditions in specific animals or populations. They may be trigger based on exposure of a venue or event to animals exhibiting the pathology (e.g., malady illness, disease, symptom, disability, defect, etc.)

A veterinary module 272 may operate similarly to a pharmacy module 270 in tracking, evaluating, analyzing, and so forth against (e.g., according to) certain criteria. Those criteria may be established by an owner, a buyer, a seller, an association, a commercial enterprise, a government regulatory body, a voluntary certification agency or group, or the like.

For example, criteria may be established by an auction, a government body, an owner, a purchase order of a prospective buyer, or the like. States, federal governments, specific agencies therein, or officers thereof may all establish criteria for verifying or certifying that an animal may be processed by transport, sale, slaughter, other disposition, or the like.

The veterinary module 272 may rely on an evaluation executable to verify compliance with certain criteria, to analyze data in order to select or determine an animal suitability under certain criteria established. Thus, the veterinary module 272 may be responsible for predicting a risk, a statistical probability, a typical result, expected result, or the like.

As a direct result of evaluating data collected from records regarding an animal, population, pathogen, event, venue, or the like, the veterinary module 272 may provide reminders for action, and may record compliance with those recommendations or with the criteria established. Such compliance may be effected by a veterinarian or a caregiver, under the supervision of a veterinarian. Recording such compliance may include recording information regarding an animal identifier, an animal record, a venue for the compliance, the personnel associated with the compliance, any medicaments or preventives applied, any inspections made, or the like. It may document these all by creating or updating a database record associated with an animal, personnel, an event, a venue, an owner, an operation, a ranch, a holding facility, or the like.

Referring to FIG. 14, while continuing to refer to FIGS. 13-16 and to FIGS. 1-16 generally, a process 260 may be typical of the pharmacy module 270, the veterinary module 272, or others of the modules 262-272. In general, the pharmacy module 270 may implement a process 270 illustrated in FIG. 14. For example, a module 270, 272 may also reflect or implement a process 270, 272. Thus, it is proper to speak of any of the modules 262-272 as the software executables implementing a corresponding procedure, but also as the procedure or process step (or protocol 270, regimen 270) itself.

Typically, the pharmacy module 270 may implement a pharmaceutical process 270 of FIG. 14. This may include tracking 273 the record and history an animal, a population, or the like. Tracking 273 may involve maintaining records, updating records, analyzing records, and providing selected information from records to other entities with a need to know. Similarly, the evaluation of the tracking process 273 may also result in inputs to an animal record, reflecting comparisons, judgments, or processing of data relating to an event, administration of a protocol, administration of a medicament or preventive, or the like.

Likewise, tracking 274 of events may involve tracking 274 the analysis after or as a consequence of application of a protocol at the event or because of it. Events maybe planned or accidental, good or bad, long or short. The existence of certain populations at a location at a time or over a period of time, or the like may trigger analysis, warnings, action, or the like as a consequence of a later discovered pathology in an animal that was present in a population at the event. Typically, the event tracking process 274 may involve analyzing the events occurring, the actual protocols applied, the exposures of different animals and populations to one another, as well as the exposures of animals or populations to administration of medicament, inspections, protocols, preventives, quarantines, holding pens, feeds, and so forth.

Thus, the event tracking process 274 may involve analyzing information, inputting information into records of animals, personnel, venues, events, and so forth for future references, and extracting information from the records in order properly analyze, evaluate, judge, rate, notify, track, evaluate risk, and communicate in response to queries about or detection of outbreaks of pathologies.

In general, a calendering process 275 may involve evaluating records of medicaments, preventives, quarantines, and the like. In conjunction with and reliance on (or interaction with) records of animals, populations, events, and the like, a calendering process 275 may be responsible to analyze records, extract certain data, evaluate that data, and recommend entry or output of other data.

Data may be specifically directed to scheduling of administration of protocols, medicaments, preventives, quarantines, or other treatments, observations, or regimens in accordance with the analysis. Typically, exposure to a pathogen at a certain time implies observation over some other subsequent period of time in order to observe any possible contagion, symptoms, or the like. The calendering process 275 may be responsible for evaluating timing of actions, protocols, observations, and the like in order to properly effect administration of a medicament, preventive, quarantine, or the like in order to limit the spread of a pathogen, evaluate the contagion of a pathogen, evaluate the effects or risks of a pathogen exposure, or the like. Risk may be based on statistical analysis of times, pathogens, populations, breeds, venues, or other measurable factors.

Typically, a notifying process 276 or step 276 may be responsible for extracting information from records corresponding to animals, populations, personnel, events, venues, and the like. For evaluating that information it may need to extract key information (data from fields) from the records searched. It may then begin preparing notifications and recommendations to go out to other animal holders such as those entities associated with records.

For example, if a detection of a pathogen, or some specific pathology in an animal, occurs, then two types of information may be critical. One is that required to assess a source of the pathogen or pathology. An illness, disease, weakness, or the like in an animal may result from genetics, exposure to a bacterium or virus, infection from a venue or another animal, an injury, or the like. Accordingly, notifying owners or caretakers of animals, venues, personnel, and the like may be important and urgent. Identifying these animals to which an animal has been exposed upstream or prior to detection of a pathology may be important to assessing what the pathology is, and where it originated.

Likewise, warning all animals situated similarly to the animal in which the pathology is detected may be just as critical. Thus, notification backward up the contact stream of an animal may be important and forward notification of all other animal owners whose animals were similarly exposed to the same upstream venues, events, animals, and personnel may be important. Thus a notifying module 276 may be responsible to review data from records, evaluate that data, and select other records and the entity associated therewith for notification. It may formulate a notification protocol to provide important information to limit disease, limit exposure, and begin closing in on and closing down a pathology that may be contagious.

Typically, the pharmacy module 270 may implement the steps 273, 274, 275, and 276 within itself as to medicaments, preventives, quarantines, and the like. Typically, however, administering 277 is outside the purview of a pharmacists. Thus, the pharmacy module 270 may make analyze data about populations, pathogens, and remedies to also make recommendations for the administering 277 of a protocol, medicament, preventive, or quarantine.

Typically, only a veterinarian or an owner or other caregiver will actually be administering 277 that recommendation. Thus, a veterinary module 272 may actually carry through to the administering 277, of a specific protocol, medicament, preventive, quarantine, or the like.

In reality, a computer may execute everything in the pharmacy module 270 including providing a shipping label and shipping a product on a schedule to a veterinarian, owner, or caregiver. Thus, the process 270 may be implemented on a computer controlled shipping and packaging system that inventories, evaluates, recommends, and ships on a schedule or in response to an event-specific instruction. It may control preparation and distribution, on schedule, any recommended or authorized medicaments, preventives, or the like in order to effect a protocol recommended for an animal or population.

Ultimately, reporting 279 is important in order to maintain accurate and complete records. Thus, the reporting step 279 may involve reporting back to the data base 84 of records of animals, personnel, venues, organizers of events, through the computers associated therewith, information to be added to records corresponding thereto. Thus, a record of an animal, a population, a venue, a location within a venue, a specific pen or holding corral, a veterinarian, a caregiver, an assistant, a pharmacist, an auction, a market, a packing house, or the like may be updated or have its records updated, or have records of animals it has processed updated by the reporting 279 of information evaluated and deemed appropriate for updating those records.

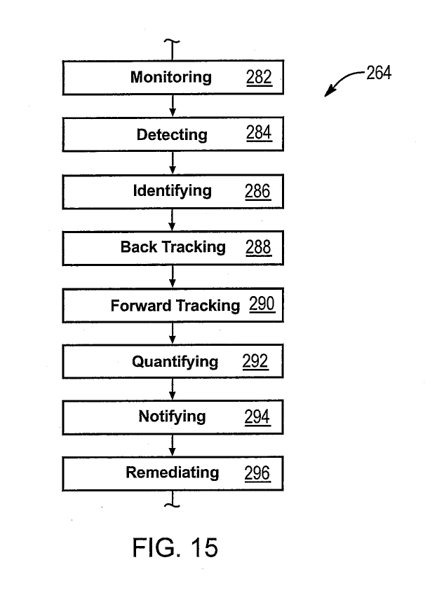

Referring to FIG. 15, in one embodiment, a certification process 264, may be implemented by a certification module 264, or other certification-type module such as the regulatory compliance module 262, permitting module 266, auction module 268, market or packing module 269, or the like. This may be done in a series of steps 282-296 implemented as a process. The process maybe controlled, directed, instructed, tracked, evaluated, analyzed, and generally implemented by a system of executables 256 hosted on one or more processors 14 on one or more computers 12. Typically, all of the software modules 262-272 may be implemented on processors 14 interconnected over the internet 72 or another internetwork 72.

In the illustrated embodiment, monitoring 282 may involve selecting records 90 of animals, venues, events, personnel, veterinarians, pharmacies, care givers, assistants, owners, auctions, packing houses, carriers, dairies, or others who process animals in any way. Monitoring 282 may involve establishing criteria, and selecting records 90 based on those criteria. Following selection, the records may be evaluated and data may be extracted from fields in those records and analyzed according to other criteria. Thus, a processor 14 may be responsible to implement a software module 262-272 to evaluate records, in order to select records, and extract from records data from various fields in those records according to criteria for selection of records and criteria for selection of information to be extracted.

After extracting information, the monitoring step 282 may be responsible to process (e.g sort, analyze, compare, evaluate, etc.) the information extracted in order to determine if certain other criteria are met. For example, criteria may be received for certifying that an animal is sufficiently low risk of, or free of pathogens and pathologies specified. The step 282 is responsible for determining that an animal has been isolated from certain populations exposed to pathogens for a pre-determined amount of time. It may also otherwise monitor the condition, exposures, and symptoms of individual animals, populations, and the like.

Likewise, the same situations may be monitored or the same characteristics may be monitored for a specific venue, for events, and for personnel. Thus, pathogens or pathologies detected in animals at any point may be tracked back to personnel who attended or worked with the animals. This may include veterinarians who attended the animals, pharmacies who provided medicaments or preventives administered to the animals, and so forth. IT will typically include any compositions or protocols (regimens) administered.

Typically, monitoring process will involve a continuing process of selection of records, extraction of data, analyzing and otherwise processing of data. It may, and very valuably so, be responsible for linking of animals, populations, venues, events, and personnel by contacts with animal sand pathologies of concern.

In order to obtain certifications required for permits, for transports, other regulatory compliance, sale at auction, grading for packing, or the like, the monitoring process step 282 may be relied upon to search, evaluate, compare, and process any and all connecting (contacting) records 90. It may then recommend ratings that pass certification, or rank certification and grading by a specific criterion or at a threshold in order to meet some particular grading level, risk level, and so forth.

The detecting step 284 may involve detecting within a record, as described hereinabove, a value of a particular entry in a particular field. That value may be important because it causes alarm, requires remediation, investigation, inspection, or the like, or simply requires reporting. Accordingly, detecting 284 is typically directed to a pathology that has been flagged or identified according to certain criteria. If a certain disease precludes an animal from being sent to a slaughter house or packing plant, that fact needs to be evaluated. Certain reports and recommendations must be forwarded to the owner, and to any one else having a transaction involving the animal. It may warrant reporting to those operating an event or protocol that has involved or will involve the animal.

Thus, detecting 284 may typically be directed to evaluating records according to certain criteria, selecting those records for evaluation according to other criteria, and then evaluating data extracted from the records according to established (e.g. private, public, government, etc.) standards in order to detect, verify, or otherwise identify a pathology, a pathogen, or the like.

A pathology may involve a birth defect, hereditary defect, disease, injury, infection, or other medical condition caused by a bacterium, virus, or the like. Thus, a pathology may be associated with a pathogen such as a bacterium, virus, parasite, infection, or the like or some other cause. It may simply be an injury Likewise, a pathology may be associated with a hereditary defect, and so forth.

After detecting 284 a pathology or pathogen to which an animal is subject, identifying 286 involves identifying contacts. Typically, initially, identifying 286 current contacts (e.g. exposures, proximity, touching shared space, water or feed, etc.) of an animal is a first step. Again, contacts may involve other animals, other populations, care givers, veterinarians, other attendants, pharmacies, chemical compositions, auctions, venues, events, and so forth. Depending on the virulence (e.g. strength, communicability, consequences) of a particular pathology, identifying 286 may be important to timeliness of such warning and remediation may need to be directed to warning, quarantining, treating, observing, or otherwise applying a protocol to a venue, a personnel individual or group, certain animals, populations, holding pens at a venue, an event, or the like.

Often, a pathology in an animal is not particularly virulent, and treatment is most important. Warnings upstream and downstream in the contact chain with such an animal maybe less important. Nevertheless, highly virulent pathologies may be extremely important to immediately contain, and to track back to a source. Effective control may require contacting forward to any contacts, but these may be cut short by quarantine. Typically, although forward contacts may be limited by quarantining an animal, a population, or the like, notification is important in defining the quarantine.

Backtracking 288 and forward tracking 290 are similar to identifying 286. That is, backtracking 288 involves selecting records of animals, venues, personnel, events, veterinarians, and so forth that have interacted with an animal. The principal purpose of backtracking 288 is suitable analysis to address and assess past contacts, past contact times, time periods, populations, individual animals, and the like that may have been a source for a pathogen or pathology detected 284 in an animal.

Thus, those contacts may have occurred simultaneously with a group of animals at an event, such as a roundup, treatment, show or auction. They may simply be animals held or processed at a particular location or venue at which the animal in which the pathogen or pathology was detected 284 was once present. Typically, those venues or events may have held populations over some period of time.

Personnel may have attended, applied protocols, treated, or otherwise interacted with animals. Thus they may be sources, or may have records that will connect to other animals treated simultaneously with the animal in which a pathogen or pathology is detected 284. Thus, backtracking 288 may analyze data to provide a principal benefit of identifying a source of a pathology or pathogen that has been detected 284 in a particular animal.

Nevertheless, by backtracking 288, the stage may also be set for evaluating records, analyzing data, and determining contacts with other animals, in order that the owners or caregivers of those other animals be notified of the detecting 284 of the specific pathology or pathogen of concern.

Forward tracking 290 may be analogous to backtracking 288. Nevertheless, forward tracking involves following forward with all contacts, persons, events, times, locations, populations, quarantines, personnel, individual animals, and the timing thereof of an animal in which a pathology or pathogen has been detected 284. Often, forward tracking 290 is cut short by quarantining. That is, if a record is analyzed a history of an animal may show a disposition toward, or a record of, a pathology or pathogen, then, depending on the risk factors analyzed (e.g. contagion transmissibility, consequent risk to an animal's health or life, commercial risks, and so forth. Forward tracking 290 may then be reduced to quarantining immediately. No forward contact is permitted with additional animals by any animal subject to such a pathology.

Nevertheless, forward tracking 290 may also be applied to animals identified during after backtracking 288. For example, a pathology may be tracked through data analysis of records 90 back from an animal in which a pathology is detected to a different population, event, venue, personnel, or the like. The objective is to identify a time, place, condition and animal that originated, or exposed the subject animal to, the pathology. Forward tracking may then begin at that backtracked record 90 in order to evaluate additional records and prepare risk assessments forward of that source. This analyzes animals and entities similarly situated of the animal from which the source was back tracked. It facilitates warnings and risk assessments to care givers, owners, and the like.

The numbers of contacts may be formidable. Nevertheless, by having computer systems 10, 70, 84, 256, 260, 200 supporting and implementing the processes 264, large amounts of data may be reviewed, selected, extracted, evaluated, analyzed, generated, reported, and updated. The evaluation of risk may be computationally programmed to involve pathologies, numbers of animals, and exposure times, and thus provide statistical analysis and quantification of risk. A human being is simply not capable of mentally doing so, nor of tracking the records, keeping the records, or forwarding the information. Tracking and linking, assessing and reporting may occur based on rapid navigation and processing of data.

Quantifying 292 the risk presented by a pathogen may also be referred to as risk assessment 292. Here, the number of contacts, the time of contacts, the time period of contact, the virulence of the pathogen detected, or the like may be factored into a risk assessment. Likewise, time since exposure, or time of exposure, may factor into probabilities of infections by or infections of other animals.

Formulae may be developed, statistically for quantifying the risk of injury, loss, infection, or other consequences of a pathology, as well as the basic risk of development of the pathology itself. Thus, both the risk of infection, for example, as well as the risk of injury, loss, or death, may be calculated statistically, based on evaluating records over the history of various animals and populations.

Similarly, any outbreak may be monitored, and statistical data may be collected from the records of the animals involved in those populations, and observed and recorded in detail. Accordingly, risk profiles may be developed for herds or populations of animals, individual animals, strains of animals, breeds of animals, locations, personnel, and even venues events. Thus, veterinarian's practices may lead to records reflecting a higher risk of certain pathologies in animals attended by that veterinarian. Similarly, a specific auction or event may develop a statistical risk, like a reputation. Thus, a certification process 264 may literally analyze rate, and rank any of the foregoing entities as to the risk each presents for a pathology.

Once quantified 292, any particular pathology may be added to a record, 90. Such data may be distributed by notifying 294 in the records of any entities that need to know. This may include a source of the pathology, or a potential target of the pathology. Thus, backtracking up the time line, to animals, events, venues, personnel, and the like that were exposed to an animal, as tracking 290 downward to those that are contacted by an animal subject to pathology will be equally important for notifying 294 all such entities. Inputs or updates may be received through their records in their computers. The result may include quantification 292 of a risk of a particular pathology as a result of the detection 284.

Any record of any entity that has been so notified 294 may then be updated, notified, queried, and the like. This may be done by broadcast generally, but is most effective when point-to-point to provide certification 264 or not of a particular animal, population, event, or the like.

In some circumstances, notifying 294 will simply be a precursor to remediating 296 the pathology. Remediating will typically be the application of a protocol, such as a surgery, administration of a medicament, administration of a preventive, or quarantining of an animal or population. Venues may be quarantined, events may be quarantined, personnel may be quarantined, and so forth.

Typically, the animal husbandry world is not dominated by stories of any “typhoid mary” veterinarians or animal attendants. Similarly, animal populations, not being as carefully tracked as individuals, and not being able to speak, do not have a reputation for carriers who carry a disease or pathology but do not show the symptoms thereof.

Nevertheless, in a system 256 and processes 260, 264, 300 in accordance with the invention, detection through careful analysis by computer processing of records may identify carriers of genetic defects, pathogens, and the like who do not necessarily display symptoms thereof.

In contrast, pathogens such as mad cow disease preclude large populations from being processed into the meat packing industry. These may be isolated more quickly, identified promptly, and remediated. Populations may be certified 264 to be free of such pathologies by a process 260, 264, 300 in accordance with the system 256 of the invention.

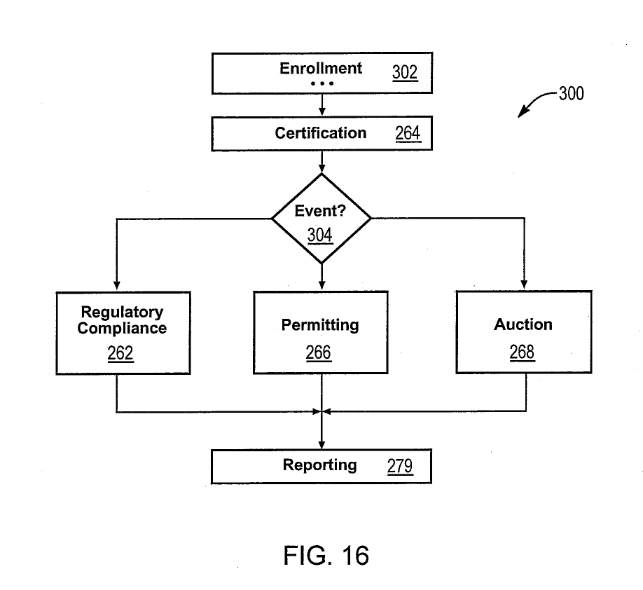

Referring to FIG. 16, a process 300 for implementing a herd management system 70 in conjunction with any type of certification module 262, 264, 266, 268, 269, may be implemented by enrolling 302 an individual animal, a population (herd), or a venue, such as an auction, a slaughter house, a packing plant, a ranch, a farm, an owner, or the like.

Following enrollment 302, software may be loaded on computers of the enrolled entity or otherwise corresponding to the enrolled entity.

For example, a company may host software in accordance with the invention on computers in accordance with the system 70 described hereinabove. Accordingly, records 90 may be created in the database 84 of the system 70 as described hereinabove with respect to FIG. 4. Similarly, a mobile application system 130 may be implemented (loaded, programmed, etc.) onto mobile computing devices of owners, caretakers, attendants, and the like corresponding to the entity venue, event, and so forth.

Ultimately, by enrollment 302 may be included implementation of hardware, software, data uploading, and so forth corresponding to the entity enrolled 302. Thereafter, certification 264 may include any or all of the processes 260, 264, 30 as well as implementation of any of the software modules 262-272.

Accordingly, certification 264 may take the form of certifying by the processes 260, 264, 300 of FIGS. 14-16 according to the risk factors and the verification of those risk factors for the entity. Ultimately, a test 304 may determine what type of event is to be undertaken. Again an event may be associated with sale, transport, slaughter, care, harvesting, shearing, milking, or otherwise deriving animal products from an individual or a population of animals. Depending on what type of event is to be undertaken, the test 304 may direct a user to the regulatory compliance module 262, permitting 266, an auction 268, or the like (see FIG. 12). For example, also included may be the options such as certifying for market 269 or packing 269, or the like.

A veterinary office is also a venue, and the veterinary and the attendants or assistants at that clinic are also entities that may be subject to certification 264. Thus, in addition to regulatory compliance and permitting or commercial activities such as auction, market sale of products and byproducts, packing of meat, and so forth, may be included veterinary visits, regular veterinary attendance of pharmaceutical interventions. These last may include medicaments, preventives, administration of supplements, or the like. Thus, certification 264 may be applied to any entity desiring it, on behalf of any entity willing to require it or willing to acknowledge and provide some benefit to those entities that have certification 264 compared to those that do not.

Thus, for example, certain permitting 266 and regulatory compliance 266 may be required or no movement of an animal, no sale, no processing, or the like is allowed. In contrast, in other commercial environments, wool from sheep that have been certified according to some standard set of criteria may command a better price for their wool. Cows that have been certified 264 according to certain criteria may command a better price for milk, and animals that have been certified according to certain criteria may command a better price for meat. Thus, the test 304 may determine what modules 262-272 may be executed to implement the processes 260-264.

Reporting 279 may be in response to the need to provide a permit, a certificate, ranking, rating, pricing, verified statement, independently audited statement, or the like regarding the condition of an animal, a population, a product, a byproduct, or the like. Thus, reporting 279 may include a response to a query of a buyer seeking to purchase a population of animals in an online or other auction.

Similarly, reporting 279 may be in response to a query by a regulatory agency seeking to verify whether a permit may issue for transporting livestock. Similarly, an inspector may test by querying online a database 84, thus being able to determine or verify that certain animals have passed certain standards, have met the criteria, or otherwise have qualified under certain evaluations. Those evaluations may be conducted on the records through a query resulting in a reporting 279 of the analysis. Thus, selections may be made based on the reporting 279 and the processing of the modules 262-272.

Thus, in general, data may be kept regarding pathogens and pathologies, such as names, times, locations, identifications of individuals and populations exposed thereto, as well as conventional data such as incubation times, contagion, remedies, and the like. Also, statistical risks or ratings for susceptibility or risk of infection, risk of loss, risk of death, and the like may also be included and associated with pathogens or pathologies that have been detected, traced, or otherwise identified and analyzed by a system in accordance with the invention.

Similarly, individual animals may include in their records certain data, certain evaluations, and the results of processing of that data to identify exposure dates, ranges of exposure, contact animals identified both upstream and downstream, to which a subject animal has been exposed. Similarly, the details of quarantines including times, places, and the like may be provided. Similarly, processing may include evaluating criteria and analyzing the exposure dates and ranges against quarantine detail according to a pathology or pathogen being analyzed.

A system in accordance with the invention such as the system 256 executing the processes 260, 264, 300 may analyze the data in the animal records 90 or population records 90, or even event records 90, venue records 90, personnel records 90, and the like. It may analyze risk, analyze exposures, clarify quarantine details and exposure dates in order to minimize or quantify 292 risk presented to others exposed to the entity to which the record 90 pertains.

Likewise, pharmacological histories including medicaments, supplements, schedules, preventives, and the like administered to an animal, a population, or at a venue, or the like may be recorded, and may be subject to analysis according to criteria for selection by a slaughter house, an auction, a state or federal agency, an association, a buyer, or the like.

Likewise, genealogies of animals may be included in the records and thereby analyzed in order to place statistical data, quantify 292 risk, and otherwise rate, rank, or classify an animal, population, or the like.

In certain embodiments the pharmacy module 270 may actually keep records 90 on populations, animals, products, shipping, and schedules. This may be used not only as a record 90, but may be analyzed by a processor 14 in accordance with the invention in order to determine a schedule for application of medicaments, preventives, supplements, or other interventions. These may be supplied according to the schedule. Thus the pharmacy module 270 may provide shipping manifests, shipping labels, shipping instructions, and delivery of products to a venue, personnel, or the caretakers or owners of an animal population in order to facilitate more readily, and almost automatically, the administration 277 of pharmaceutical interventions.

The administering of veterinary interventions may be handled similarly. Thus, populations, animals, products, shipping schedules, shipping modes, shipping carriers, and the like may be subject to certification processes 260, 264, 300, and may be implemented, automated, and controlled the processors 14 with the analysis of the records 90, according to selected criteria.

Thus, a certification may be an independent certification, private certification, individual buyer certification, or some regulatory or commercial requirement or standard, such as a state permit, state or federal regulation compliance, packing plant standards, or commercial auction requirements. Certain standards may be documented and accepted by an association, a government, an individual, or other entity.

Similarly, such standards may be documented and catalogued thereby being subject to citation. Thus, a government standard, industry standard, or purchase order specification may have a citation for the standard, as well as a recitation or the standard or an abbreviated reference to it by title. Accordingly, the processes 260, 264, 300 may implement the standard by obtaining criteria or a checklist including dates, events, compliance, certificates, verifications, signatures, sworn statements, and the like.

Such verifications and certifications may involve dates of inspections, dates of administrations 277, identifications of agents responsible, including all personnel and attendants, as well as identification of protocols, medicaments, preventives, supplements, and those administering them. These may be processed with the history of an animal in order to analyze animals to select those that pass certain criteria, or to rank animals, populations, venues, or events according to their risk presented to those who come in contact with them.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A computerized, livestock-management system comprising:
a first processor corresponding to an entity exercising central control and responsibility for a first population of livestock;
a first memory device, comprising a computer-readable storage medium, operably connected to the processor and containing a first database comprising first records corresponding to the entity and the livestock;
a first input device operably connected to the first processor;
a first output device operably connected to the first processor;
a second processor, operating at a remote location and disconnected from the first processor;
a second input device operably connected to the second processor and delivering inputs corresponding to an event at the remote location and a second population of livestock, a subset of the first population, corresponding to the event and located at the remote location;
a second output device operably connected to the second processor to output information corresponding to the second population;
a second memory device, operably connected to the second processor and storing a second database, comprising a copy of the first database, having altered records corresponding to the second population and altered based on inputs from the second input device at the remote location; and
the second processor, programmed to selectively connect to and disconnect from the first processor based on an availability of an Internet connection therebetween;
the second processor, further programmed to upload altered data, corresponding to the altered records, when connected to the Internet; and
the first processor, further programmed to synchronize the first database and second database with respect to one another when the Internet connection is available between the first processor and the second processor.

2. The system of claim 1, wherein:
the first memory device stores a plurality of first modules executable on the first processor; and
the second memory device stores a plurality of second modules executable on the second processor.

3. The system of claim 2, further comprising:
a third processor configured like the second processor to operate with respect to the first processor; and
a third memory device operably connected to the third processor and storing a third database, copied from the first database and altered in accordance with an event at a third location, remote from the first location and corresponding to a third population, a subset of the first population.

4. The system of claim 3, wherein the first processor is further programmed to communicate with the second and third processors, when connected respectively to the Internet.

5. The system of claim 4, wherein the first modules further comprise:
a synchronizing module executable to broker updates between the first database and the second database;
a listening module operable to receive over the internet an upload from the second database by the second processor.

6. The system of claim 5, wherein the synchronizing module further programs the first processor to broker updates between the second database and the third database.

7. The system of claim 6, wherein the second memory device stores, for execution by the second processor, a mobile application programmed to read at least one of the second database, an RFID device, a bar code reader, and a GPS device.

8. The system of claim 7, wherein the mobile application is programmed to read the second database, RFID device, bar code reader, and GPS device.

9. The system of claim 8, wherein the mobile application is programmed to read from and write to records in the second database, including at least one of:
an animal record corresponding to an animal in the second population;
an event record corresponding to an activity and the second population at the remote location;
an attendant record corresponding to an operation conducted at the event;
a herd record corresponding to the second population;
a herd record corresponding to the first population;
a geographical record corresponding to at least one of the animal, the first population, the second population, the event, the attendant, and the remote location;
a product record corresponding to a product administered at the event;
an enterprise record corresponding to ownership of at least one of the first and second populations;
a facility record corresponding to a facility corresponding to the enterprise; and
a personnel record corresponding to at least one of the animal, the first population, the second population, the event, a previous event, the enterprise, and the facility.

10. The system of claim 10, wherein the mobile application is programmed to read from and write to records in the second database, including at least three of:
an animal record corresponding to an animal in the second population;
an event record corresponding to an activity and the second population at the remote location;
an attendant record corresponding to an operation conducted at the event;
a herd record corresponding to the second population;
a herd record corresponding to the first population;
a geographical record corresponding to at least one of the animal, the first population, the second population, the event, the attendant, and the remote location;
a product record corresponding to a product administered at the event;
an enterprise record corresponding to ownership of at least one of the first and second populations;
a facility record corresponding to a facility corresponding to the enterprise; and
a personnel record corresponding to at least one of the animal, the first population, the second population, the event, a previous event, the enterprise, and the facility.

11. A method for managing livestock, the method comprising:
providing a first processor;
providing a first memory device comprising a computer readable storage medium storing data and modules executable on the first processor to operate on the data;
providing the first memory device, wherein the data includes a first database, comprising at least one of
animal records corresponding to a first population of animals and a second population, the second population being selected from the first population and located at a remote location away from the rest of the first population,
an event record corresponding to at least on of a treatment of the second population administered at the remote location and a previous treatment,
a geographical record comprising at least one of a map, GPS coordinates, a name, an address, and a unique identifier corresponding to a previous treatment corresponding to an animal of the second population,
an enterprise record corresponding to ownership of the first population,
a facility record corresponding to at least one of the treatment and the previous treatment,
a product record corresponding to a product administered at least one animal of the animals at the treatment and the previous treatment,
an attendant record corresponding to at least one of a veterinary staff and a care staff corresponding to at least one of the treatment and the previous treatment, and
a report corresponding to at least one of the first population, second population, the at least one animal, the treatment, the previous treatment, the product, the enterprise, and the event;
providing a second processor in a mobile device, located at the remote location and disconnected from the first processor when so located;
providing a second memory device, storing a mobile application and a copy of the database and being operably connected to the second processor to load thereinto the mobile application; and
conducting the event based on presentation by the mobile application of old information from the copy of the database; and
uploading to the copy of the database, by the mobile application, new information corresponding to the second population and the event.

12. The method of claim 11, further comprising:
connecting the first and second processors to the internet;
connecting the second processor to the first processor when a connection is available over the Internet; and
brokering, by the first processor, updating the database with the new information from the copy of the database.

13. The method of claim 12, further comprising:
providing a second mobile device;
providing to the second mobile device a second copy of the database distinct from the first copy of the database;
providing, by the second mobile device, while operably disconnected from the first processor, additions to the second copy of the database; and
uploading from the second mobile device the additions.

14. The method of claim 13, further comprising:
connecting the second processor to the first processor over the internet;
downloading to the second memory device a new copy of the database, distinct from the copy and containing the additions;
disconnecting the second processor from the first processor;
treating a third population, a subset of the first population, based on the new copy of the database, while the second processor remains disconnected from the first processor; and
uploading, by the second processor, changes to the first database reflecting the treating of the third population.

15. The method of claim 14, further comprising brokering by the first processor an acceptance of the additions and the changes to the database, based on at least one of timing and a hierarchical protocol.

16. The method of claim 11, wherein:
the first processor is programmed for
logging in a user;
loading data corresponding to at least one of a user and the second population;
receiving a request to complete an exchange of data with the second processor;
parsing the request;
complying with the request;
generating a response; and
updating the database when the response corresponds to uploading new information directed to records in the data and received from the second processor.

17. The method of claim 16, wherein the second processor is further programmed for:
logging in a user;
initializing a mobile application based on the copy of the database;
selecting a mode of operation;
setting up an event;
processing livestock based on the event and the copy of the database; and
altering the copy of the database while disconnected from the internet;
waiting until an internet connection is available; and
uploading to the first processor, when the internet connection is available, alterations made by the second processor to the copy of the database while disconnected from the first processor.

18. A computerized, livestock, certification system comprising:
a first processor executing a database engine servicing a database, the database storing first records corresponding to an a first population of livestock;
a first memory device, comprising a computer-readable storage medium, operably connected to the first processor and containing modules executable on at least one of the first processor and a second processor operably connected to the first processor over a network, the modules comprising
a certification module programmed to analyze the first records,
a herd management module
the first processor operably connected over a network to a second memory device, operably connected to a second processor and storing a second database, comprising a copy of the first database, having altered records, corresponding to a second population selected from the first population, the altered records being altered based on inputs from the second input device at a remote location away from the first processor; and
the first processor, programmed to selectively connect to and disconnect from the second processor based on an availability of an Internet connection therebetween;
the first processor, further programmed to upload the altered data, when connected to the Internet; and
the first processor, further programmed to analyze the altered data and provide a warning corresponding to the records corresponding thereto in the database;
the first processor, further programmed to provide a certification of an animal based on the analysis of the altered data.
